(19) United States
(12) Patent Application Publication
(10) Pub. No.: US 2017/0296753 A1
(43) Pub. Date: Oct. 19, 2017

(54) HANDHELD MEDICAL SUBSTANCE DISPENSING SYSTEM, APPARATUS AND METHODS

(71) Applicant: Accunit, LLC, Los Altos, CA (US)

(72) Inventors: Garrett ROWE, Los Altos, CA (US); Howard S. SUTKIN, Los Gatos, CA (US); Dustin Alan COCHRAN, Boulder Creek, CA (US); Charles Madison SKINNER, Scotts Valley, CA (US)

(73) Assignee: Accunit, LLC, Los Altos, CA (US)

(21) Appl. No.: 15/374,235

(22) Filed: Dec. 9, 2016

Related U.S. Application Data
(60) Provisional application No. 62/323,795, filed on Apr. 18, 2016.
(60) Provisional application No. 62/412,793, filed on Oct. 25, 2016.

(51) Int. Cl. A61M 5/315; A61M 5/31

(52) U.S. Cl. CPC A61M 5/31515; A61M 5/31513; A61M 5/31526; A61M 5/31558; A61M 5/3156; A61M 5/3157; A61M 5/31581; A61M 5/3137; A61M 2205/36; A61M 2205/3317; A61M 2205/3303; A61M 2005/3152; A61M 2005/3142; A61M 2005/3139; A61M 2005/3126; A61M 2205/3606; A61M 2205/50; A61M 2205/502; A61M 2205/52; A61M 2205/582; A61M 2205/583; A61M 2210/0606; A61M 2205/586

(57) ABSTRACT

Various handheld medical dispensing configurations and methods including the following: Handheld medical dispensing system comprises a syringe, which has a plunger, releasably mounted to syringe actuation apparatus, the apparatus having an actuator configured such that it can move the plunger more than once to dispense a plurality of amounts of substance from the syringe and having a housing adapted for singled handed administration of an injection.

### RELATED APPLICATIONS

This application is a non-provisional of U.S. Provisional application Nos. 62/412,793 filed Oct. 25, 2016 and 62/323,795 filed Apr. 18, 2016, the entirety of both of which are incorporated by reference. This application also incorporates the following applications by reference: U.S. application Ser. No. 14/481,708 filed Sep. 9, 2014; Ser. No. 14/286,701 filed May 23, 2014; and Ser. No. 13/550,473 filed Jul. 16, 2012; and provisional application 62/323,795 filed Apr. 18, 2016, the entirety of each of which is incorporated by reference herein.

### BACKGROUND OF THE INVENTION

In the field of plastic surgery, non-invasive procedures have enjoyed increasing popularity over the past ten years. The most common procedures include injection of liquid tillers in facial lines and wrinkles as well as the use of agents which paralyze selective muscles of the face in order to provide diminishment and smoothing of wrinkles. Typically, a dermal filler or paralytic agent such as Botox.®.Cosmetic (onabotulinumtoxinA), is used on a recurrent outpatient basis. Such agents typically are available in sterile vials of product in freeze-dried form. The clinician will reconstitute the preparation according, to a specific recipe of sterile saline (fluid) added to the vial of product. Once mixed, the preparation is drawn up into a syringe, for subdermal delivery using a small bore medical needle. The most common delivery method uses a one cc disposable syringe which is fitted with a disposable needle. Importantly, small, reproducible allotments of the fluid are delivered via multiple injections into the muscles of the face, such as the frontalis muscle in a field of the face which would benefit from treatment.

Further, it is well known that the use of small syringe during injections of the face on a subject who is sitting in a chair is generally uncomfortable for the health care provider and subject to unsteady maneuvering of the syringe due to the necessity of pushing on the plunger which is located at the rear end of the syringe assembly, while trying to hold the syringe steady. Accordingly, there remains a need to provide a handheld dispenser or syringe that can be more stable or desired tiller or fluid amounts from a handheld dispenser or syringe in such applications in order to provide predictable and/or desired results.

### SUMMARY OF THE INVENTION

The present invention involves improvements in medical substance delivery systems and can be used in the delivery of any substance.

In one variation, the delivery system includes a dispensing unit for use with a syringe, the syringe having a barrel, and a plunger slidable relative to the barrel to dispense substances located therein.

For example, such a medical dispensing system can include a housing assembly being configured to secure the barrel of the syringe such that a distal end of the barrel is adjacent to a distal end of the housing assembly; a drive mechanism coupled to the housing assembly, the drive mechanism comprising a plunger fitting that engages the plunger; an actuator; and a drive assembly comprising a plurality of linkages coupled between the plunger fitting and the actuator, at least a first linkage being coupled to the actuator and moveably coupled to a pivotable linkage, such that movement of the actuator in a single continuous stroke moves the first linkage relative to the pivotable linkage causing the pivotable linkage to pivot in a first direction and subsequently in a second direction in the single continuous stroke of the actuator, such that movement of the pivotable linkage in the first direction produces movement of the plunger fitting in a rearward direction relative to the distal end of the housing and movement of the pivotable linkage in the second causes movement of the plunger in a forward direction.

In another variation the dispensing system include a dispensing unit having a locking surface being moveable for releasably locking the syringe therewith. In one example, the improved syringe can comprise a syringe body comprising a reservoir located therein; a retaining member extending along a length of the syringe body and extending away from the syringe body, the retaining member comprising a retention surface wherein engagement of the locking surface against the retention surface releasably locks the retaining member and syringe body to the dispensing unit preventing movement of the syringe body relative to the dispensing unit, and where the retaining member distributes a force applied by the locking surface along the length of the syringe body to reduce distortion of the syringe body.

In another variation the syringe can have a portion of the retention surface that is parallel to an axis of the syringe body. The retaining member can comprise a flange shape, the flange shape comprising at least one slot extending parallel to an axis of the syringe body, where at least a side of the at least one slot that defines the retention surface. A variation of the flange can comprise a planar surface extending parallel to an axis of the syringe body.

The syringe retaining member can have one or more slots, where the slots can be linear or arcuate shaped. In another variation the retaining member extends along at least a half of the length of the syringe body. The retaining member can be detachable from the syringe body or the retaining member can be integrally formed with the syringe body and is detachable by breaking the retaining member along a weakened area.

In another variation, the proximal end of the plunger member comprises a tapered shape. The retaining member can comprise a planar surface extending parallel to the axis of the syringe body. In another variation, the retaining member comprises a thickness less than a diameter of the syringe body.

Another variation of the system can include a dispensing system with a syringe comprising: a syringe body comprising a reservoir and plunger slidably located therein; a retaining member extending along a length of the syringe body and extending away from an axis of the syringe body, the retaining member comprising an opening having a retention surface, the retention member configured to distribute a force applied to the retention surface along the length of the syringe body to reduce distortion of the syringe body; and a housing assembly comprising: a moveable locking surface, the locking surface enlargeable against the retention surface to releasably lock the retaining member to the housing assembly to prevent movement of the syringe body relative to the dispensing unit; a drive mechanism coupled to the housing assembly, the drive mechanism comprising a plunger fitting that engages the plunger; an actuator; a plunger fitting configured to receive a portion of the plunger; and a drive assembly coupled to the actuator, such that movement of the actuator in a single continuous stroke moves the plunger fitting relative to the distal end of the housing.

The dispensing unit can further include a drive assembly having a plurality of linkages coupled between the plunger fitting and the actuator, at least a first linkage being coupled to the actuator and moveably coupled to a pivotable linkage, such that movement of the actuator in a single continuous stroke moves the first linkage relative to the pivotable linkage causing the pivotable linkage to pivot in a first direction and subsequently in a second direction in the single continuous stroke of the actuator, such that movement of the pivotable linkage in the first direction produces movement of the plunger fitting in a rearward direction relative to the distal end of the housing and movement of the pivotable linkage in the second causes movement of the plunger in a forward direction.

In another variation,

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail. Variations of the access device and procedures described herein include combinations of features of the various embodiments or combination of the embodiments themselves wherever possible.

### BRIEF DESCRIPTION OF DRAWINGS

FIG. 1 is an exploded diagrammatic view of a handheld dispensing system tor apparatus) according to one embodiment of the invention.

FIG. 2 diagrammatically illustrates a side elevational view of the embodiment of FIG. 1 assembled with the syringe mounted on the system's syringe actuation apparatus of FIG. 1 and the actuation apparatus pusher member spaced from the syringe plunger.

FIG. 3 diagrammatically illustrates the system of FIG. 2 with the illustrated lever arm partially depressed to have the pusher member engage the syringe plunger thumb portion and advance the syringe plunger so as to eject a small amount of fluid form the syringe needle to prepare the apparatus for use.

FIG. 4 diagrammatically illustrates the system of FIG. 3 from the opposite side and after the lever arm has been released and allowed to return to its start or rest position as shown in FIG. 2 and being advanced toward a target site on the skin of the patient.

FIG. 5 diagrammatically illustrates the system of FIG. 4 after an operator or physician has penetrated the patient's skin with the needle and fully depressed the lever arm to engage the lever stop member to dispense a desired volume or a preset and/or predetermined volume of substance from the syringe.

FIG. 6 diagrammatically illustrates the system of FIG. 5 alter the system needle has been removed from the patient and the lever arm released and allowed to return to its start or rest position as shown in FIG. 4.

FIG. 7A is a diagrammatic partial sectional view taken generally along line 7A-7A in FIG. 2.

FIG. 7B is a top diagrammatic view of the housing illustrated in FIG. 7A showing the housing sidewall edges that are secured to the syringe actuation apparatus frame or base.

FIG. 7C is a top diagrammatic view of the stop device or assembly for the lever arm depicted, for example, in FIG. 1.

FIG. 7D is a diagrammatic enlarged partial sectional view of the release mechanism generally diagrammatically depicted in FIG. 7A.

FIG. 7E diagrammatically illustrates the push button and lever arm of 7D rotated 180 degrees.

FIG. 7F diagrammatically illustrates a transverse cross-sectional view taken generally along line 7F-7F in FIG. 7D.

FIG. 8A diagrammatically illustrates a ratchet mechanism, generally shown in FIG. 7A, for advancing the pusher member.

FIG. 8B diagrammatically illustrates the mechanism of FIG. 8A with the ratchet wheel of FIG. 8A rotated and the pusher member advanced.

FIG. 8C diagrammatically illustrates the mechanism of FIG. 8B with the ratchet wheel of FIG. 8A further rotated and the pusher member further advanced.

FIG. 8D diagrammatically illustrates the mechanism of FIG. 8C with the pawls moved inwardly and the pusher member being reset or retracted.

FIG. 9 diagrammatically depicts one method of holding die system of FIG. 2 with a single hand.

FIG. 10 illustrates an isometric view of a variation of a dispensing unit that is configured to produce a negative pressure within a barrel or reservoir prior to delivering the contents of the substance in the reservoir.

FIGS. 11A and 11B show respective front and rear views of the device 100 of FIG. 10.

FIGS. 12A to 12E shows an example of the gear drive assembly during movement of the actuator through the first stroke segment.

FIG. 12F shows disengagement of the back drive gear mechanism.

FIG. 13 shows a bottom view of the variation shown in FIG. 10.

FIGS. 14A to 14C illustrate movement of the actuator to cause advancement of the plunger fitting.

FIGS. 15A and 15B show another variation of a device where the barrel moves relative to the plunger to generate a negative pressure.

FIG. 16 shows another variation of components for use with the injection systems including a housing and circuitry.

FIGS. 17A to 17E illustrates another variation of a dispensing unit having a modified housing that improves the ability of a user to administer a single-handed positioning and actuation of the unit.

FIGS. 18A and 18B illustrate an example of how the housing of FIG. 17A-17E facilitates one-handed operation of the injection unit.

FIGS. 19A to 19C illustrate various views of another variation of a housing where various marked portions of the housing intended for engagement by a user's hand or finger.

FIG. 20 illustrates another related concept that includes a sensing unit for use with conventional syringes and/or injection systems of the present disclosure.

FIG. 21 illustrates another related concept that includes a holding unit for use with conventional syringes and/or injection systems of the present disclosure.

FIGS. 22A to 22E illustrate an additional example of an injection systems that employs a linear rail driven by an actuator without the need for fully rotational gears.

FIG. 23A shows an example of a gear drive assembly for use with the variation shown FIGS. 22A-22E.

FIGS. 23B to 23G show an example of the injection system drawing rearward and then advancing the plunger fitting.

FIGS. 24A to 24C show an example of a locking switch used to temporarily disable the reverse feature of the device.

FIGS. 25A to 25C illustrate an improved syringe having a retaining member.

FIGS. 26A and 26B illustrate a variation of a dispensing unit similar to the variations described herein with the additional feature of having a locking surface for engaging the retaining member.

FIGS. 27A to 27C show an example of a syringe with a retention member inserted into the dispensing unit.

FIGS. 28A-28D illustrate an example of the dispensing unit locking into engagement with the syringe.

FIGS. 29A to 29C show another variation of a dispensing unit

FIGS. 30A to 30C illustrate a filling process of the dispensing unit and preparing the unit for dispensing of a substance in the syringe.

FIGS. 31A-31C illustrate the dispensing unit of FIG. 30A with the body of the unit hidden to better illustrate a drive mechanism of the unit.

FIGS. 31D to 31F illustrate an example of the drive mechanism of the dispensing unit of FIG. 30A.

FIGS. 31G to 31I illustrate an example of a one-way locking mechanism of a plunger of the dispensing unit.

FIG. 32A shows another variation of a dispensing unit that employs a constant force spring/clock spring.

FIGS. 328 to 32H illustrate the various stages of the drive mechanism of the dispensing unit of FIG. 32A.

### DETAILED DESCRIPTION

Before the present invention is described, it is to be understood that this invention is not intended to be limited to particular embodiments or examples described, as such may, of course, vary. Further, when referring to the drawings like numerals indicate like elements.

According to one embodiment of the invention, a handheld medicinal or medical substance delivery system (or apparatus) comprises actuation apparatus, which can be a mechanical actuation apparatus, and a medicinal or medical dispenser (e.g., syringe) where the dispenser is mounted or coupled to the actuation apparatus such that one can manually manipulate the actuation apparatus to deliver a substance any suitable pharmaceutical product or agent used in the treatment of facial lines and wrinkles such as a paralytic agent,) from the dispenser to a patient site or target site (e.g., muscle layer immediately beneath the skin) or to each of a plurality of patient sites or target sites, while holding the substance delivery system. The actuation apparatus can be configured to provide the operator flexibility in an amount of substance to be delivered by, for example, allowing the operator to determine the extent the actuation apparatus is actuated. For example, the operator need not fully actuate the actuation apparatus so that a smaller amount or volume of substance is dispensed. The actuation apparatus also can be configured to allow the operator to deliver a desired, preset or predetermined amount or volume of the substance. It also can be configured to facilitate repeatable delivery of the same desired, preset or predetermined amount or volume of substance, which can facilitate delivery of reproducible precise amounts or volumes of substance from the dispenser. A precise amount can, for example, correspond to a drop of liquid, where the size of the drop can depend on the density of the liquid and the surrounding pressure, or a value such as 0.1 cc of substance. In contrast, delivery of precise reproducible amounts or volumes of substance may be difficult if possible with a traditional method of holding a syringe in one hand and depressing the plunger with the other hand or the thumb of the same hand.

Further, the actuation apparatus described in the preceding paragraph can be provided with an optional adjustment mechanism to facilitate delivery of different desired, preset or predetermined amounts (or volumes) of substance or different doses or dosages.

The substance delivery system also can be configured to be held and used with a single hand (e.g., in a manner that resembles holding a pen), which can enhance controllability and stability of the system during use. Further one handed operation of the system enables its user (e.g., operator or physician) to use his/her free hand to do something else. For example, when using the system to treat wrinkles, one handed operation allows the user's free hand to hold the patient's skin and stretch the skin in the areas of wrinkles, which facilitates the proper placement of the delivered agent with the other hand. Thus, with this configuration, a single hand can be used (or only one hand is needed) to hold the agent or substance delivery system, maneuver it to the desired site, direct it to penetrate the syringe needle at the desired site, and manipulate the syringe actuation apparatus to inject a desired amount of the agent or substance into the patient. In one embodiment according to the invention that facilitates such use, actuation apparatus includes a lever actuator having one end adjacent to or radially spaced from a distal portion or the distal end of the syringe barrel and its other end coupled to the actuation apparatus. This can enhance one handed control of the device. For example, a bottom portion of the delivery system can be held in the web space of the hand between the index finger and thumb and the user's index finger used to depress the lever actuator. The lever actuator end that is adjacent to the distal portion or end of the syringe barrel can be in non-overlapping relation to the exposed portion of the needle so as not to interfere with inserting the needle into the patient. Further, the lever can be pivotally mounted to the apparatus such that the user can squeeze the lever with one hand, while holding the system with that hand to dispense substance from the dispenser. With this lever configuration the user may be able to sense back pressure and control delivery rate of the substance being dispensed. Since the lever arrangement can enhance one's ability to use the device with a single hand, it can improve its ease of use. Further, the one-handed aspect can enhance stability of the system during use.

The syringe actuation apparatus also can be configured so as to be reusable. For example, it can be configured so that the dispenser or syringe is releasably or removably mounted or coupled thereto so that, for example, a used dispenser or syringe can be replaced with a new dispenser or syringe. Further, the actuation apparatus can be configured to be coupled to an off-the-shelf medical dispenser or syringe.

According to one embodiment of the invention, a handheld medicinal or medical substance dispensing system comprises a syringe (or dispenser), which can have a barrel containing a medicinal or medical substance, releasably or removably mounted or coupled to the base or frame of actuation apparatus. The actuation apparatus includes a pusher (or slide) slidably mounted to the base or frame of the actuation apparatus and a lever that can be displaced to actuate or move the pusher. The lever can be arranged with an end located adjacent to a distal end or end portion of the syringe barrel, which can be fitted with a needle. The system can include a mechanism (e.g., a stop positioned at a preset location to limit the maximum displacement of the lever from a start position) such that when the lever is fully displaced from the start position, the lever actuates the pusher (or slide) to move the syringe plunger in the syringe barrel a specific distance, which is reproducible (e.g., when the lever is again fully displaced from its start position). In one variation, the specific distance that syringe plunger travels, which can be the same distance that the pusher travels, can be adjusted, and the adjusted distance reproducible using, for example, an adjustable mechanism to preset the adjusted maximum lever displacement. For example, the pusher and syringe plunger travel distance can be preset using an adjustable stop that provides an adjustable limit on the maximum displacement of the lever from its start position so that the preset plunger distance and dispensed substance can be varied.

This specific syringe plunger travel distance, which cart be preset based on, for example, the position of the lever stop, can translate into a known and again, reproducible volume of substance or material being discharged from the syringe as described above, which can enable the user to repeatedly deliver a desired, preset or predetermined amount of substance (e.g., a fluid or paralytic agent) to, for example, the face of a patient to treat facial lines or wrinkles. The system also may be used to repeatedly deliver a dosage of the substance described above to treat facial lines or wrinkles. The dosages can be varied as well. In general, the lever, pusher, and plunger travel amount(s) or distance(s), movement amount(s), or displacement(s) can be preset e.g., through a lever stop, to dispense, deliver, eject, or inject a desired, preset or predetermined amount(s), volume(s), dose(s) or dosage(s) of substance, For example, the maximum lever displacement can be preset so that the pusher and/or plunger move a desired, preset or predetermined distance to dispense a desired, preset or predetermined amount of substance from the syringe.

Further, the system can be configured so that it can be supported by one hand, which can enhance its stability during use and the lever arrangement can enhance one's ability: to use the device with a single hand and improve its ease of use.

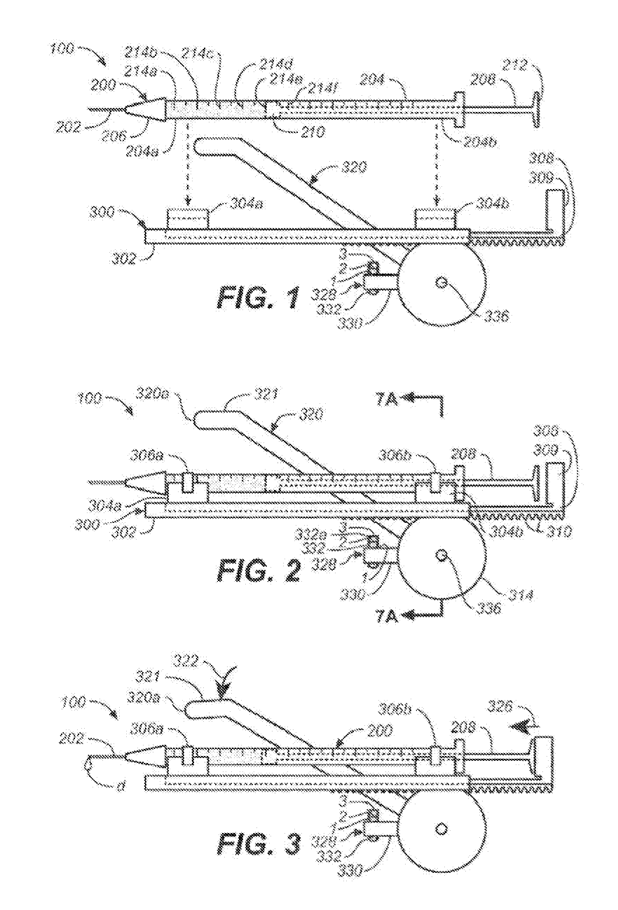

Referring to FIGS. 1 and 2, an illustrative example of one embodiment of the invention is shown. FIG. 1 is an exploded view of handheld medical substance dispensing, delivery, ejecting or injecting system 100, which includes syringe 200 and syringe actuation and/or metering apparatus or device 300. FIG. 2 shows syringe 200 releasably or removably coupled or mounted to syringe actuation apparatus 300.

Syringe 200, which can be any standard hypodermic syringe having a hypodermic needle 202 to inject fluid (e.g., a liquid or gas) into body tissue, a barrel or tube 204 to which the needle is coupled. and a plunger 208 slidably moveable in barrel 204.

Returning to FIG. 1, barrel or tube 204 has a distal end portion 204a and a proximal end portion 204b, holds fluid (e.g., any known suitable paralytic agent or dermal tiller to treat facial lines or wrinkles), and has an orifice at one end for the fluid to be dispensed therefrom into needle 202. Plunger 208 has a piston head 210 that fits snugly in barrel 204, and thumb actuator or handle 212 that can be pushed to dispense fluid from the barrel and needle. The barrel can be provided with a distal tapered tip 206 to which the needle is attached or fined and it can be transparent so that the amount of fluid or substance therein can be monitored, Syringe barrel 204 can have graduated marks or indicia 214a, b, e, d, e, f . . . n to indicate the volume of fluid therein. In the illustrative example, the volume between any adjacent two of these lines (e.g., 214d and 214e) corresponds to 0.1 cc. The marks between these lines further indicate 0.05 cc quantities. Any suitable material can be used to make the barrel such as plastic or glass.

Although one syringe configuration is shown, any suitable syringe configuration can be used as would be apparent to one of ordinary skill in the art. For example, a syringe without a needle, but to which a needle can be fitted, e.g., a disposable needle, can be used.

Before mounting the syringe to the base, the operator or physician can perform an air removal procedure as is known in the art and tap the syringe while holding it with the needle up and then pushing the syringe plunger to dispense some liquid. Alternatively, the operator or physician can use syringe actuation apparatus 300 to remove air from the syringe as will be described in more detail below.

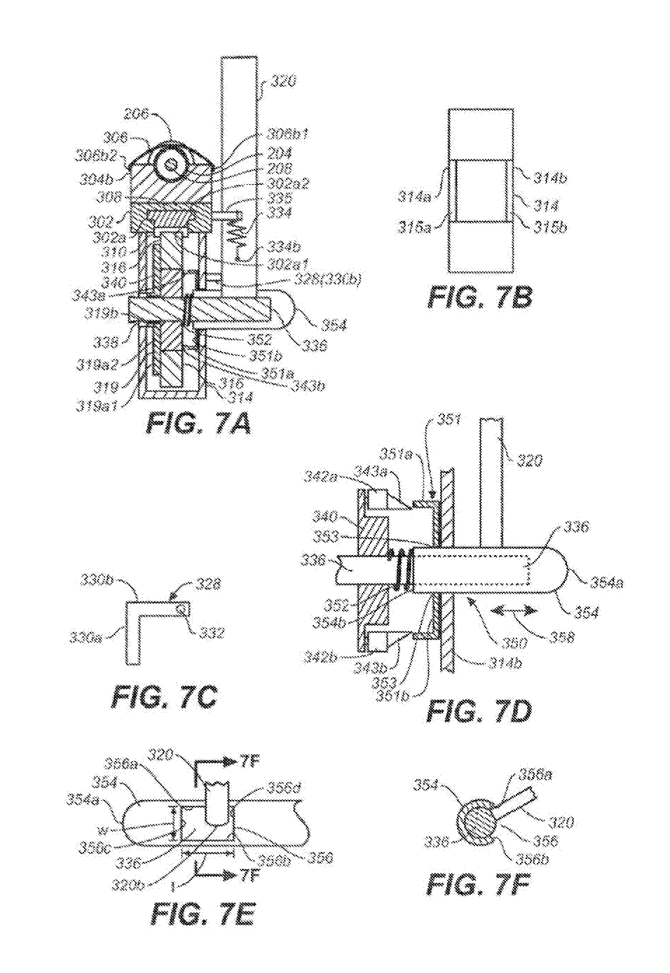

Syringe 200 is releasably or removably coupled or mounted to syringe actuation or metering apparatus or device 300. In the illustrative example, syringe actuation apparatus 300 includes a base or frame 302 to which syringe supports 304 are attached. Alternatively, the frame and supports, which can be any suitable material such as plastic, can be integrally formed as a single piece construction. As shown in FIG. 7A, support 304b has a curved surface (which can, for example, be a concave or U-shaped surface) in which syringe barrel 204 is cradled. Support 304a, which is hidden from view in FIG. 7A, has the same configuration. Although one curved surface is shown formed in support 304b any other suitable surface for supporting the syringe can be used.

Syringe 200 can be secured to syringe actuation apparatus base 302 using any suitable means. In the illustrative example, straps 306a,b secure syringe barrel 204 to supports 304a,b (see e.g., FIGS. 2 & 7A). Since each strap is secured to a respective support in the same manner only securement of strap 306b will be described for simplification. Strap 306b has a first end 306b1 fixedly secured to a support 304b along a support side) and a second end 306b2 that is detachably secured to support 304b (e.g., the other side of the support) so that the strap extends over the syringe barrel and snugly holds it against the support so as to preclude or minimize relative axial movement therebetween. Any suitable detachable securing mechanism can be used to detachably secure the second end 306b2 to the support. For example, hook and loop fasteners, which can he Velcro.®. brand hook and loop fasteners, can be used. A band of hook fasteners can be provided along one side of a strap to engage with a band of loops provided along a portion of the support. Alternatively, a band of loop fasteners can be provided along one side of a strap to engage with a band of hooks provided along a portion of the support. In one variation, both strap ends can be detachably secured to their respective support. It also should be understood that although two supports are shown, one support, two supports, more than two supports, or no supports can be used. For example, a curved surface can be formed in base 302 or no curved surface provided in which case a securing mechanism is still used to secure syringe 200 to syringe actuation apparatus 300.

Syringe actuation apparatus 300 also includes an actuator that can move syringe plunger 208 toward syringe needle 202 to dispense or inject a plurality of desired volumes or predetermined volumes of substance. In the illustrative example, one such actuator is shown and includes pusher or pusher member 308, lever or lever arm 320, and a drive or drive assembly coupling pusher member 308 and lever arm 320. A mechanism to vary the desired volumes or predetermined volumes of substance to be dispensed also can he included as will be described below with reference to the exemplary embodiment shown in FIG. 1. It should be understood, however, that although one actuator configuration will be described, other actuator configurations that can move syringe plunger 208 in syringe barrel 204 toward syringe needle 202 to dispense or eject a plurality of desired volumes or predetermined volumes of substance can be used as well as other mechanisms to vary the desired volume or predetermined volume of substance to be dispensed or injected into the patient.

Referring to FIGS. 1 and 7A, base 302 has a longitudinal channel or slot 302a formed therein and in which pusher 308 is slidably mounted such that it can move generally parallel to plunger 208 of syringe 200 when the syringe is mounted to base 302 as shown in the drawings. Slot 302a can extend the entire length of base 302 and can have a configuration that cooperates with the configuration of pusher 308 as shown, for example, in FIG. 7A, such that pusher 308 is maintained in the slot and does not fall out from base 302 when oriented, for example, as shown in FIG. 7A. In the illustrative example, slot 302a has a first portion 302a1 adjacent its opening with a first width and a second portion 302a2 inwardly positioned from the first portion with a second width greater than the first width. Pusher 308 has a corresponding first width adjacent its teeth 310 and second width that forms lateral extensions that mate with the wider portion of slot 302a as shown in FIG. 7A. Although one slot-pusher configuration has been shown, it should be understood that other configurations or mechanisms can be used to maintain pusher 308 in the slot formed in base 302. For example, when ratchet wheel housing 314 is secured to base 302 and secures ratchet wheel 316 in engagement with pusher teeth 310 as will be described below, ratchet wheel 316 can maintain pusher 308 in the slot formed in base 302 in which case pusher can have a single width. However, a slot configuration as described above can be helpful in assembly of the apparatus and avoid the need for the ratchet wheel to hold up the pusher. Housing 314 also can comprise separate components that are assembled around the mechanisms to be mounted therein as would be apparent to one of ordinary skill in the art. It also should be understood that housing 314 may have other configurations than the circular one shown. For example, housing 314 can be square or rectangular with one side open for attachment to base 302.

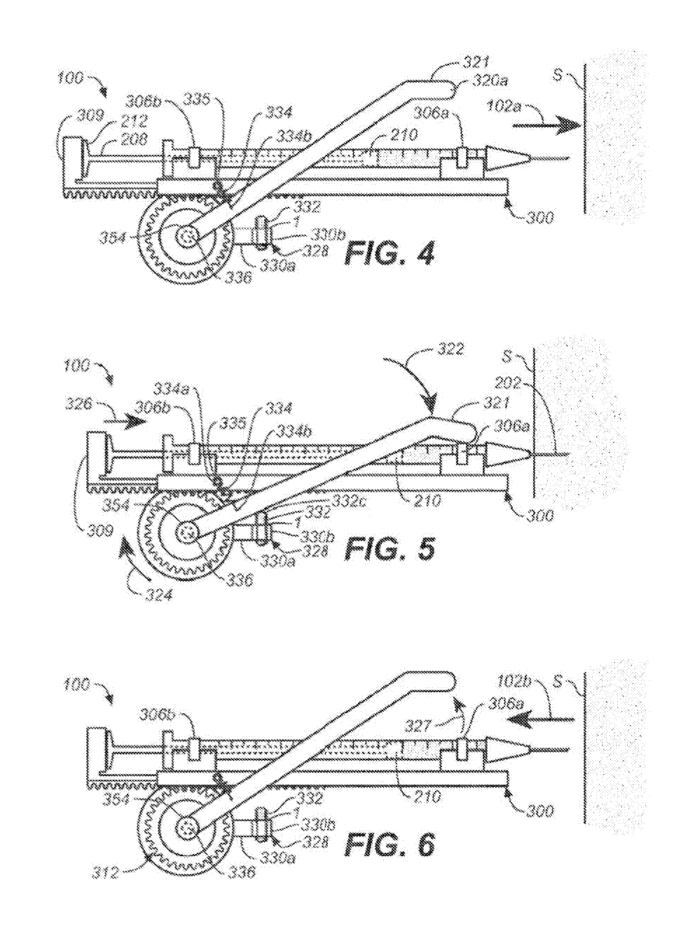

Pusher teeth 310 extend from the bottom of pusher 308 and along its length to cooperate with driving gear teeth 318 of ratchet wheel 316 so that ratchet wheel 316 can move or drive pusher 308 toward plunger handle or plunger thumb actuator portion 212 of syringe plunger 208 or move pusher 308 and plunger portion 212 therewith when pusher 308 and plunger portion 212 are in engagement as shown in FIG. 3. Pusher 308 includes a portion or extension 309 that extends in a direction opposite from pusher teeth 310 and is arranged or aligned to engage and press against plunger handle or plunger thumb portion 212 of syringe plunger 208. In this manner, pusher portion 309 advances the plunger in syringe barrel 204 when pusher 308 is moved toward the distal or needle end of syringe 200. For example, in FIG. 4, apparatus 300 is in a state where pusher 308 is not advancing plunger 208 as system 100 is moved toward a patient treatment site as shown with direction arrow 102a. Needle 202 is then penetrated though the skin “S” of a patient at treatment site as shown in FIG. 5, after which lever 320 is fully depressed in the direction of arrow 322 rotating ratchet wheel 316 in the direction of arrow 324 to advance pusher 308 and plunger 208 therewith in the direction of arrow 326 (FIG. 5) to inject medicinal substance into the patient. After the injection, system 100 can be retracted or removed from the patient as indicated with direction arrow 102b (FIG. 6) without moving the ratchet wheel or plunger. This can be repeated at one or more different sites to deliver a desired amount or predetermined amount of substance to the patient at each site.

Any suitable known ratchet assembly can be used as part of the drive to rotate the drive in one direction such as a ratchet assembly having a ratchet wheel having inner ratchet teeth and outer gear teeth combined with spring loaded pawls.

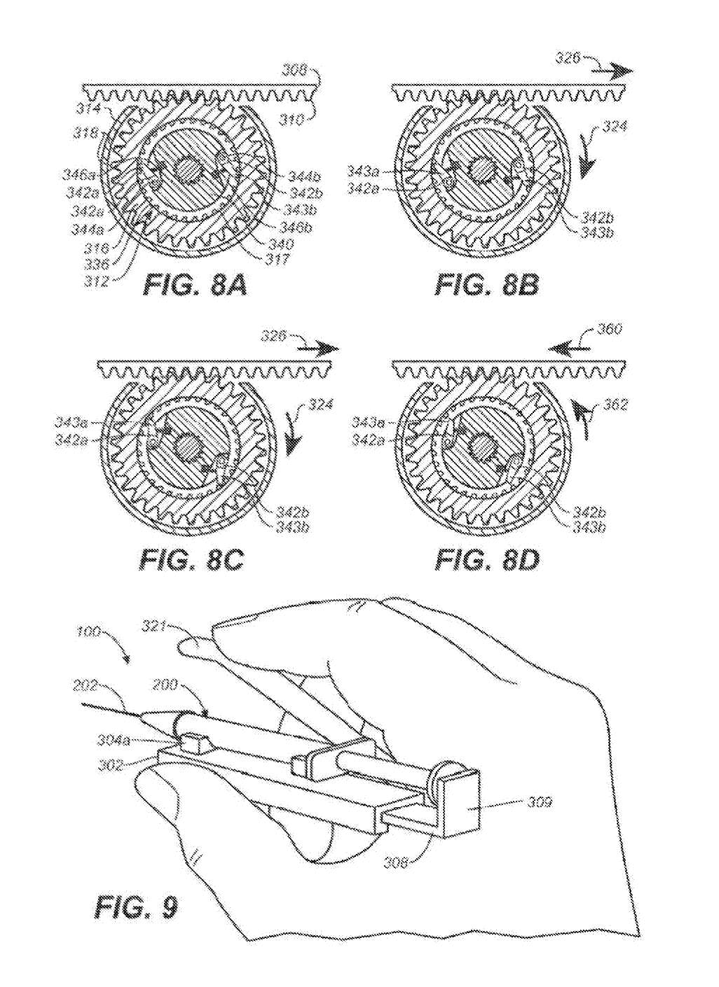

Referring to FIGS. 7A and 8A-B, one drive or drive assembly using a ratchet mechanism is shown in the illustrative embodiment for purposes of example. In this example, lever arm 320 is coupled to pusher 308 through a drive or drive assembly. The illustrated drive or drive assembly includes ratchet mechanism 312 (see e.g., FIG. 8A), which generally comprises ratchet wheel 316, which also serves as a driving gear wheel, and pawls 342a,b will be described in more detail below. In brief lever 320 turns or actuates ratchet wheel 316 to drive pusher 308 toward the distal end of syringe 200.

Ratchet mechanism 312, which is housed in ratchet housing 314 (see e.g., FIG. 8A) with axle or pin 336 extending therefrom to lever arm 320 (FIG. 7), includes ratchet wheel 316 with inner ratchet teeth 317 extending along an inner annular surface thereof. Ratchet wheel 316 also has outer gear teeth 318, which cooperate with pusher teeth 310 to drive pusher 308 in the direction indicated with arrow 326 as shown in FIG. 8B and in this manner forms a circular gear. Inner ratchet teeth 317 cooperate with ratchet pawls 342a,b, which are pivotally coupled to or are supported on pawl support 340, which is fixedly secured to pin or axle 336, which extends through a center hole formed in pawl support 340. Ratchet pawls 342a,b are pivotally mounted to pawl support or wheel 340 on pins 344a,b (see e.g., FIG. 8A), which are secured to pawl support 304 and spring loaded with springs such that they are biased or urged toward inner teeth 317 to allow ratchet wheel 316 to rotate in only one direction (see e.g., arrow 324 FIG. 8B). In the illustrative embodiment, coil springs 346a,b are placed in recesses in support 340 and arranged to bias or urge the pawls toward inner teeth 317. It should be understood, however, that other spring arrangements or configurations can be used including ones without a recess or ones using other types of springs as would be apparent to one of ordinary skill in the art. Further, pawls 342a,b are made to include ramp or wedge portions 343a,b to assist in releasing the pawls from the ratchet wheel as will be described in more detail below. In operation, when lever 320, which is fixedly secured to pin or axle 336, is depressed, it moves in the direction indicated with numeral 322 in FIG. 5 to actuate the drive assembly. That is, lever 320 rotates pin or axle 336, which is fixedly secured to and rotates pawl support 340, which rotates pawls 342a,b therewith. As pawls 342a,b move with rotating pawl support 340, they push the ratchet teeth 317 with which they are engaged and biased against to rotate ratchet wheel 316 in the direction indicated with arrow 324 as shown in FIG. 8B. As ratchet wheel 316 rotates in this direction with its teeth 318 mating with pusher teeth 310, it drives or moves pusher 308 in the direction of arrow 326 so that pusher extension or arm 309 can engage and advance syringe plunger 208 to dispense fluid. When lever 320 is allowed to return to its start or rest position as shown, for example, in FIG. 6 (e.g., by releasing lever arm 320, lever arm spring 334, which can be a coil spring, moves toward its rest position and lifts or moves lever arm 320 in the direction indicated with arrow 327 (FIG. 6) to its start or rest position. In the example shown in FIGS. 1-7A, spring 334, which urges lever 320 toward its start or rest position, has one end 334a secured to post 335, which extends from base 302, and its other end 334b secured to lever arm 320. In this manner, spring 335 can maintain lever arm in its start or rest position when the lever arm is not being depressed. Other mechanisms also can be used to urge lever arm 320 toward its start or rest position as would he apparent to those skilled in the art.

As lever arm 320 returns to its start or rest position, it rotates pawl support 340 through pin or axle 336 in the opposite direction. As pawl support 340 rotates in that opposite direction, pawls 342a,b slide over consecutive ratchet wheel teeth 317, which in the illustrative example, are shown with an asymmetrical configuration where each tooth has a steeper slope on one side as compared to the other. It should be understood, however, that other ratchet teeth configurations can be used as well. Lever arm 320 can again be actuated to further advance ratchet wheel 316 and pusher 308 and this repeated as desired as diagrammatically shown in FIG. 8C.

Although one drive or drive assembly has been shown, it should be understood that many other drives or drive assemblies can be used as would be apparent to one of ordinary skill in the art and that the illustrative example is not intended to limit the scope of the invention.

Referring to FIG. 8D, release of Pawls 342a,b to reset pusher 308 is shown. When the pawls are moved radially inward as shown and as will be described in detail below, the user can push pusher 308 back as generally indicated with arrow 360 turning released ratchet wheel in the direction indicated with arrow 362. In this manner, the user can reset pusher 308 to a desired position (e.g., back to its position shown in FIG. 2 to ready apparatus 300 for another syringe loaded with the desired agent or substance after syringe 200 has been removed).

Regarding the coupling of lever arm 320 to ratchet wheel 316, lever arm end 320b is fixedly secured to axle 336 or in a recess formed in axle 316 using any suitable means and pin or axle 336 is fixedly secured to pawl support or wheel 340 using any suitable means (see e.g., FIGS. 7A and 7E). For example, glue, adhesive or welding can be used to secure lever arm 320 to pin or axle 336 and a splined connection can be provided between pin or axle 336 and pawl support or wheel 340. Alternatively, all of those connections can be made with for example, glue, adhesive or welding. In this manner, lever arm 320, which is biased toward its rest or start position (see e.g., FIG. 4), can be depressed to move in the direction of arrow 322 (FIG. 5) to rotate pin 336 and pawl support wheel 340 and then released so that it moves as indicated with arrow 327 (FIG. 6) and returns to its start or rest position shown in (see e.g., FIGS. 4 and 6).

Returning to FIG. 7A, pin or axel 336 extends through openings in sidewall 314a,b of ratchet housing 314. Sidewall 314a can be provided with a hearing or hushing 338 to facilitate or enhance rotation of pin or axle 336 therein. Alternatively, the materials used to make the axle and sidewalls of the housing can be selected to facilitate the desired rotation of axle 336 in sidewall 314a without a bearing or bushing. Push button 354, which will be discussed in more detail below, extends through sidewall 314b and provides a support in which pin or axle 336 can rotate. Again the materials can be selected to facilitate the desired rotation as would be apparent to one of ordinary skill in the art.

Referring to FIG. 7B, ratchet housing 314 has a cutaway portion that exposes sidewall edges 315a,b so that they can be fixedly secured to base 302 as shown in FIG. 7A.

Since ratchet wheel 316 is supported by pawls 342a,b, which are mounted on pawl support 340, which is mounted on pin 336, which is mounted in housing 314, which is secured to base 302, ratchet wheel 316 is supported in housing 314 in engagement with pusher teeth 310. Alternatively, a ratchet wheel holding mechanism can be used to maintain the ratchet wheel in the desired position as shown in FIG. 7A. In this arrangement, ratchet wheel holding mechanism 319 comprises a disk 319a having an outer portion 319a1 fixedly secured to ratchet wheel 316, an inner portion 319a2 aligned with pawl wheel 340 and not secured thereto so as to allow relative movement between inner portion 319a2 and pawl wheel 340, and cylindrical hub 319b that is rotatably mounted on axle 336 and from which disk inner portion 319a2 extends. Inner portion 319a2 also is rotatably mounted to axle 336, which extends through a central opening in inner portion 319a2. Inner portion 319a2 also can be recessed so as to be spaced from pawl wheel 340. Further, holding mechanism 319 can be formed as a single unitary element and can comprise any suitable material such as plastic.

As discussed above, lever arm 320 can be configured and/or arranged to facilitate one handed use of the system. In the illustrative embodiment, lever arm 320 has a distal end or free end 320a (FIGS. 1-6), a proximal end 320b (FIG. 7E), and a portion 321 (FIGS. 1-6), which can be used to push, actuate or depress lever arm 320 in the direction of arrow 322 (see e.g., FIG. 5). Portion 321 can be angled to extend generally parallel to the longitudinal axis of syringe barrel 204 or syringe needle 202 when the syringe is mounted to syringe actuating apparatus 300. The position of portion 321 adjacent to the distal end portion of syringe barrel 204 and its depicted configuration can enhance the ease in which a finger can depress lever 320. However, other configurations can be used. Lever arm distal end or free end 320a is arranged to move alongside barrel 204. It also is adjacent to the distal end portion of syringe barrel 204 and in non-overlapping, relation with needle 202 so as not to interfere with placement of the needle in the patient as shown, for example, in FIGS. 4 and 5 where the lever arm is shown in non-overlapping relation with needle 202 throughout a full stroke. Lever free end 320a or the distal free end portion of lever 320 also can be radially spaced from barrel 204 as shown in FIG. 7A.

In the embodiment shown in FIGS. 1-6, a mechanism is provided so that the dispensing system can deliver a desired, preset or predetermined amount or volume of substance. In one embodiment, a mechanism is provided to limit the movement or advancement of lever arm 320 (or limit the maximum arc or arc length that it can travel) in the direction of arrow 322 (see e.g., FIG. 5). In this manner, the limiting mechanism also limits the corresponding rotation of ratchet wheel 316 and translation of pusher 308. This limiting mechanism is arranged so that the travel distance or displacement of lever arm 320, ratchet wheel 316, and/or pusher 308, upon full depression or actuation of lever arm 320 from its start position, can be preset in a manner such that a plurality of desired, preset or predetermined volumes of substance can be dispensed or ejected from syringe 200. The maximum movement of lever arm 320 can be preset to drive to ratchet wheel 316 and/or pusher a preset distance. In the illustrated embodiment, a lever stop or lever travel limit device (e.g. stop or limit device 328) is provided. Stop 328 has an arm 330 that extends from ratchet housing 314 and turns 90 degrees or any suitable amount so as to extend into the path of lever arm 320. Arm 330 has a first portion 330a that extends from housing 314 and a second portion 330b that extends into the path of lever arm 320 (see e.g., FIG. 7C). The stop sets or defines the fully advanced position of lever 320 as lever 320 cannot travel beyond the stop. In another embodiment, the stop can be formed with a member that extends from base 302 and under lever 320. When the stop does not have an adjustable stop member, syringe actuation apparatus 300 can be calibrated with syringe 200 or any syringe or dispenser to be used therewith using known techniques to locate where the stop will be located or preset to provide a desired, preset or predetermined amount or volume of substance to be ejected.

Referring to FIG. 1-6, stop arm 330 of stop 328 can include an optional mechanism or member to vary the maximum displacement of lever arm 320 or the maximum stroke or maximum distance or arc that lever arm 320 travels to vary the desired, preset or predetermined amount or volume to be dispensed. In the illustrative embodiment, stop arm 330 can be provided with an optional threaded bore in which an optional stop member or set screw 332 is rotatably positioned so that set screw 332 can be raised or lowered to change or adjust the range of movement that lever arm 320 can travel. Set screw 332 has a contact surface or portion 332a that lever arm 320 contacts upon full advancement thereof where the contact portion position is adjustable since set screw 332 can be raised or lowered such that the desired, preset or predetermined amount of substance to be dispensed from the syringe can be varied. Set screw 332 can include any suitable means for assisting in rotating it to move it up or down such as a slot in its lower end or any other suitable mechanism as would be apparent to one of ordinary skill in the art.

Set screw 332 can include indicia to indicate a desired, preset or predetermined dispensing, ejecting, or injection amount or volume so that it can be positioned at a desired location or preset to provide a desired, preset or predetermined amount or volume of dispensed substance. For example, the indicia can permit the user (e.g., operator or physician) to adjust set screw 332 (the stop member), so that the amount or volume of dispensed substance can be varied (e.g., to eject 0.1 cc, 0.2 cc, or 0.3 cc from the syringe). These volumes can, for example, correspond to desired, preset or predetermined amounts or volumes of substance to be dispensed. Syringe actuation apparatus 300 can be calibrated with syringe 200 or any syringe or dispenser to be used therewith using known techniques to create the indicia and provide this result. For example, marker lines “1,” “2”, and “3” can be created by calibrating actuation apparatus 300 with syringe 200 so that 0.1 cc, 0.2 cc, or 0.3 cc are ejected from the syringe upon full displacement of lever arm 320 when set screw 332 is at certain levels and providing the marker lines on the set screw based on the results.

Thus, and with reference to, for example, FIGS. 2, 4 and 5, system 100 can be calibrated so that the position of stop member or set screw 332 can be preset as shown, for example, in FIG. 2 or 4 so that the pusher moves the plunger a desired, preset, or predetermined distance (the pusher can move the same distance to provide this result) to provide a desired, preset or predetermined ejection or 0.1 cc of substance (e.g., fluid) from syringe 200 when lever 320 is depressed from its start position as shown, for example, in FIG. 4 to a fully depressed position as shown in. FIG. 5 where the lever arm contacts set screw 332. When set screw 332 is in this position, the bottom of marker line “1” is aligned with or flush with the upper surface of stop arm 300 (see e.g. FIG. 4). Referring to FIG. 2, when marker line “2” has been created based on a calibration to provide 0.2 cc of substance (e.g., fluid) and stop member or set screw 332 adjusted or lowered so that the bottom of marker line “2” is aligned or flush with the upper surface of stop arm 330 and lever arm 320 is depressed from its start position to where it contacts stop member or set screw 332, 0.2 cc of substance (e.g., fluid) is dispensed from syringe 200. Another marker line, e.g., marker line “3” (see e.g. FIG. 2), can be provided above marker line 2 such that when its bottom is aligned or flush with the upper surface of stop arm 330, 0.3 cc of substance (e.g., fluid) is dispensed when lever arm 320 is depressed from its start position to where it contacts stop member or set screw 332.

The ability to preset the maximum displacement of lever arm 320, which activates pusher 308 to move plunger 208 in syringe barrel 204 a specific distance, which is adjustable and can be preset based on, for example, the position of lever stop member 332, can translate into a known and again, reproducible, volume of substance or material being discharged from the syringe as described above, which can enable the user to repeatedly deliver desired, preset or predetermined amounts or volumes or substance (e.g. a fluid such as a paralytic agent) to a patient. This can, for example, enhance the user or operator's ability to obtain symmetry of results or to deliver the desired agent symmetrically over a region.

It is further noted that the (1) lever arm actuator or (2) lever arm stop or stop member taken individually or in combination contribute to the ergonomics of the handheld dispensing system.

Regarding the pawl release described above, one example of a pawl release mechanism for releasing the pawls to reset pusher 308 is diagrammatically shown, for example, in FIGS. 7D-F and generally designated with reference numeral 350. Referring to FIG. 7D, release mechanism 350 generally comprises push button 354, open cylinder 351, which the push button moves to cooperate with pawl ramp or wedge portions 343a,b of pawls 342a,b, and a biasing mechanism such as coil spring 352 that biases cylinder 351 away from pawls 342a,b.

Push button or actuator 354 is a hollow tubular member having a closed end 354a and an open end 354b. Push button 354 is rotatably and slidably mounted an axle 336 and has a slot or window 356 formed therein through which lever arm 320 extends as diagrammatically depicted in FIG. 7E, which diagrammatically illustrates the push button and lever arm of FIG. 7D from the opposite side. The cut-out in push button 354 that forms slot 356 has an upper edge 356a, a lower edge 356b, and side edges 356c,d. FIG. 7F diagrammatically shows a transverse section view of the push button and lever arm of FIG. 7E and the slot upper edge 356a and lower edge 356b. The width “w” of slot 356 is sized so that lever arm 320 can travel its intended or desired distance or stroke when depressed or released. Length “l” of slot 356 is sized to permit operation and sliding of push button 354 about lever arm 320 so that the push button can be pushed to release the pawls and allowed to return to its unactuated position.

Push button 354 also extends over axle 336 through an opening in ratchet housing sidewall 314b as shown, for example, in FIG. 7D where it engages coil spring 352 and can move back and forth in the sidewall opening. Coil spring 352 surrounds axle 336 and has one end abutting pawl support 340 and its other end abutting the annular edge face at the open end of push button 354. In this manner, coil spring 352 biases or urges push button 354 toward its start position or unactuated position as shown, for example, in FIG. 71D. Open cylinder 351, which can be a shallow cylinder as shown, comprises a cylindrical wall 351a with an open end and annular wall portion 351b at its other end. Annular wall portion 351b has a center opening through which push button 354 extends and is fixedly secured as indicated with reference numeral 353. The fixed connection between annular wall portion 351b and push button 354 can be made with any suitable means such as welding, adhesive or glue that is applied, for example, continuously for 360 degrees or less than 360 degrees or is applied non-continuously in a spaced pattern. Spring 352 urges push button 354 away from pawl support 340 and through push button 354 spring 352 urges annular wall portion 351b of cylinder 351 against housing sidewall 314b. In this manner, an operator can push button 354 toward pawl support 340 to move the pawls radially inward and then release push button 354 so that is can return to its unactuated position shown in FIG. 7D and as generally indicated with reference arrow 358. Pawls 342a,b are made to include ramp or wedge portions 343a,b, which form part of pawls 342a,b. Ramp or wedge portions 343a,b are diagrammatically shown in FIG. 7D with a tapered configuration with one side sloping radially inward so that when cylinder 351a engages the ramp or wedge portions, it moves pawls 342a,b radially inward. The illustrated tapered configuration or sloping side that forms a ramp can extend the length of the ramp or wedge portions 343a,b, which can, for example, run a short distance from the distal or free end of a respective pawl along the distal end portion of the pawl, or run from the distal or free end of the pawl close to the pivot pin, or a different distance. However, it should be understood that ramp or wedge portions 343a,b can be angled, configured and/or arranged differently than that shown to cooperate with cylinder 351 such that pawls 342a,b are moved radially inward when cylinder 351 is pushed toward pawl support 340 and against such ramp or wedge portions as would be apparent to one of ordinary skill in the art. Other release mechanisms also can be used.

Referring to FIG. 7D, when the user wants to reset pusher 308, the user pushes push button 354 to move the open end of cylindrical member 351 that faces pawl support 340 toward pawls 342a,b. As the open end of cylindrical wall 351a engages the pawl ramp or wedge portions 343a,b, it pushes ramp portions 343a,b or pawls 342a,b radially inward about their pivot pins as diagrammatically shown in FIG. 8D. In this position, the user can reset the position of pusher 308. It also should be understood that other release mechanisms can be used. After resetting pusher 308, push button 354 can be released to allow the pawls to reengage ratchet wheel 316.

The following example is set forth to illustrate one method of operation of the invention, and is not intended to limit the scope of the invention. For example, the method described hereafter will be disclosed in connection with treating facial wrinkles or lines, but is not intended to be limited to such treatment. More specifically, the following example involves injection of a liquid filler or paralytic agent in a facial line or wrinkle to paralyze selective muscles of the face in order to provide diminishment and smoothing of the line or wrinkle. The injection can be repeated to treat other lines or wrinkles or to add more liquid tiller to a treated line or wrinkle.

Syringe 200, which contains a paralytic agent as described above, can be primed to remove air therefrom before it is mourned to syringe actuating apparatus 300 as described above or syringe actuation apparatus 300 can be used to prepare the syringe for use. In the latter case, syringe 200 can be secured to syringe actuation apparatus 300 and the apparatus held so that syringe needle 202 extends upwardly. Then syringe actuation apparatus 300 can be actuated by pressing lever arm 320 (e.g., with the index finger) to advance pusher portion or arm 309 into contact with the syringe proximal end portion or thumb portion 212 of syringe plunger 208 if not engaged therewith (FIG. 2), and then further actuated by further depressing lever arm 320 to advance syringe plunger 208 in syringe barrel 204 to dispense a first small amount of substance (e.g., a drop of substance “d”) to prepare the syringe for use as shown in FIG. 3. FIG. 3 shows lever arm 320 only partially depressed to dispense a small amount of substance.

If apparatus 300 has not been used to prepare the syringe for use (e.g., the syringe was prepared before securing it to syringe actuation apparatus 300) or pusher extension 309 is not engaged with syringe thumb actuator portion 212 (FIG. 2), lever 302 is depressed to engage pusher 308 with thumb actuator portion 212 and then lever 302 is released to allow it to return to its start position or at rest state as shown in FIG. 4.

With handheld system 100 ready for use, the operator or physician holds system 100 in one hand with the system cradled in the web of the physician or operator's hand between the thumb and index finger and the index finger on lever portion 321 as generally diagrammatically shown in FIG. 9. The middle finger can provide further support for holding the apparatus 300. The operator or physician uses the other hand to hold the patent's skin and stretch the skin in the areas of facial lines or wrinkles to assist in properly placing the syringe needle and delivery of the agent. The operator or Physician then advances dispensing apparatus 100 toward a target site on the patient's skin as indicated with arrow 102a in FIG. 4.

Referring to FIG. 5, after the operator or physician has penetrated the patient's skin with syringe needle 202, he or she fully depresses lever arm 320 to engage set screw or stop member 332 of lever stop 328 to move pusher 308 and syringe plunger 208 a desired (or preset and/or predetermined distance) and inject a desired (or preset and/or predetermined) amount (or volume, dose or dosage) of a paralytic agent in the muscle beneath the facial line or wrinkle being treated.

FIG. 6 shows dispensing apparatus 100 after syringe needle 202 has been withdrawn from the patient and lever arm 320 released and allowed to return to its start position as shown in FIG. 4. The operator or physician can then penetrate the needle into another site to treat other lines or wrinkles or to add more liquid filler to a treated line or wrinkle and this can be repeated until the syringe is emptied or no longer has a desired (or preset and/or predetermined) amount (or volume, dose or dosage) of substance or paralytic agent contained therein. Then syringe 200 can he removed from syringe actuation apparatus 300, pusher 308 reset, and another syringe containing the desired paralytic agent mounted to apparatus 300 to continue treatment of the patient or to treat a different patient. Further, stop member or set screw 332 of stop 328 can be adjusted after any injection to change or vary the desired amount (or volume, dose, or dosage) or the desired (or preset and/or predetermined) amount (or volume, dose, or dosage) of agent to be injected at any point during a procedure.

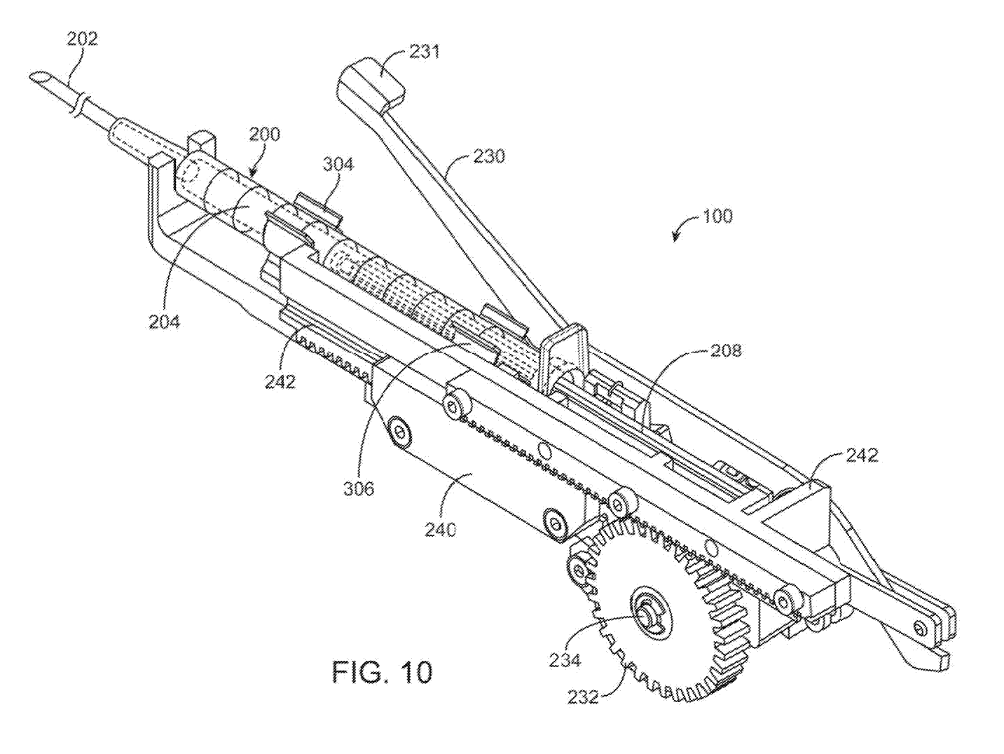

FIG. 10 illustrates another variation of a dispensing unit 100 that is configured to produce a negative pressure within a barrel or reservoir 204 prior to delivering the contents of the substance in the reservoir. The illustrated variation is also shown with the inner mechanism of the device 100 without any outer covering or housing for purposes of clarity. However, any number of coverings, shells, ergonomic fittings, etc. can he provided with the exemplary device shown. As noted below, many variations of the device include coverings that allow a medical caregiver to administer the device using a single hand. Also, additional features can be incorporated into the dispensing unit such as a feedback sensor or other circuitry that can allow for submission of a current through a needle on the syringe or an electrode coupled to the needle and/or syringe.

As shown, the dispensing unit 100 includes a syringe 200 coupled to the device 100. In the illustrated example, the syringe 200 is releasably coupled to the dispensing unit 100 via one or more coupling or securing mechanism 304, 306. Any number of securing mechanisms can be employed in the unit 100. Additionally, alternate variations of the dispensing unit 100 can include a syringe built in or integrated with the dispensing unit.

The device 100 depicted in FIG. 10 includes an actuator 230 that is mechanically coupled to a first gear 232 and a second gear 236 via respective first shaft 234 and second shaft 238 (where in FIG. 10, the second gear and second shaft are located behind a guide member 240). Additional variations of the device 100 permit the caregiver to grasp the device using a thumb and middle finger while using an index finger to operate the actuator 320. Accordingly, the housing (not shown) can be configured to be conducive to such handling. Moreover, in many variations of the device 100 the actuator 320 extends towards a dispensing end (e.g., towards the cannula/needle 202 end of the device). This configuration provides the user the ability to grasp the body or device with an index and middle finger and actuate the lever 230 with an index finger. Clearly, other holding configurations are within the scope of alternate variations of the device of the present disclosure. However, the stability of the device (upon being inserted into tissue) increases when the index finger applies pressure to the lever 230 and the point of contact 231 moves through an arc or path that is close to where the operator is grasping the unit.

FIG. 10 also illustrates a plunger 208 of the syringe 200 being coupled to a plunger fitting 242 that couples the plunger 208 to the lever via the gearing system. Accordingly, the plunger fitting 242 moves the plunger upon activation of the gear drive assembly which is triggered by the actuator 230. As shown, the plunger fitting 242 can also include various keys and channels 242 to allow for smooth advancement of the plunger 208.

The gears in this embodiment are shown as rack-in-pinion gears with the first rack gear 205 moving the plunger 208 in a rearward direction and second rack gear 212 moving the plunger 208 in a forward direction.

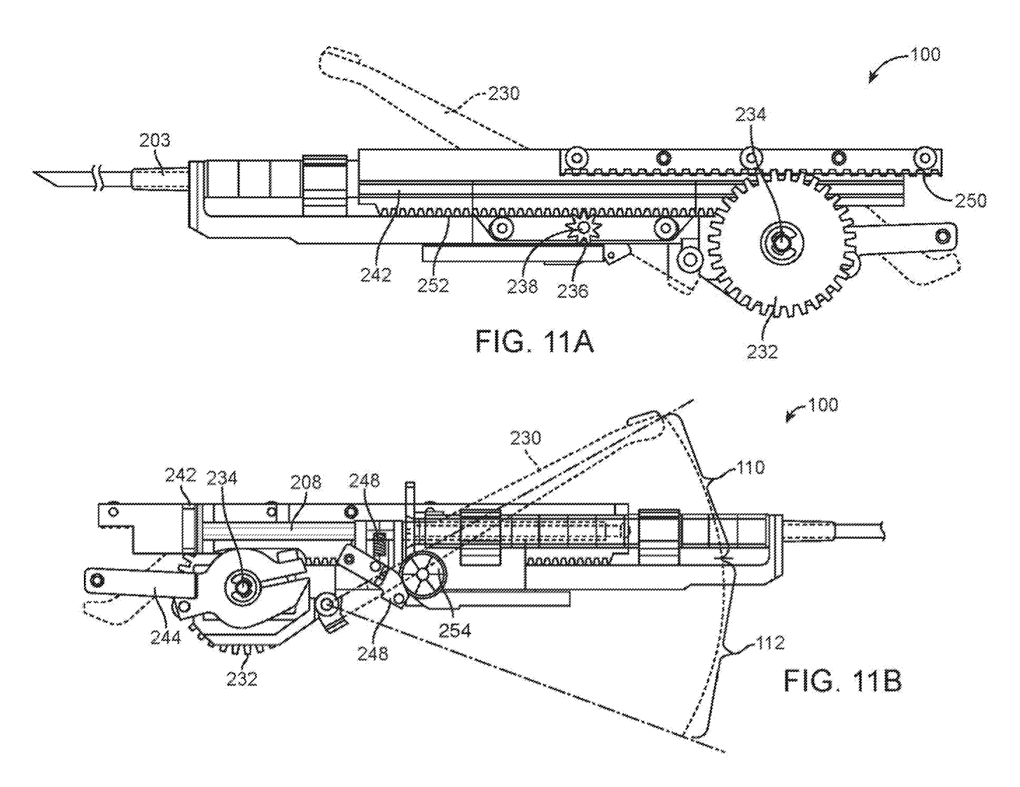

FIGS. 11A and 11B show respective front and rear views of the device 100 of FIG. 10. The lever 230 and guide member 240 (not shown) are hidden from view in FIGS. 11A and 11B to better illustrate the drive mechanism of the illustrated variation where the drive mechanism causes movement of the plunger upon movement of the lever actuator 230. As seen in FIG. 11A, a first drive gear 232 engages a first rack gear 250. The first drive gear 232 rotates in response to rotation of a first shaft 232. FIG. 11B illustrates the opposite end of the first shaft 234 being coupled to a drive lever 244. The illustrated variation also includes a back drive gear 238 secured to a back drive shaft 238. The back drive shaft 238 moves upon rotation of a back drive pulley 254.

A variation of the device permits movement of the plunger 208 in a first direction and a subsequent second direction using a single stroke of the lever 230. Typically, this dual directional movement allows a medical caregiver to insert the needle into the patient and then actuate the device 100 such that negative pressure is generated in the reservoir, which causes blood or other fluids to be drawn into the reservoir or a window 203 of the device 100. In certain cases, such as those discussed above, the medical caregiver is attempting to confirm placement of the needle in muscle or other tissue but not in a vessel. If the caregiver determines that the needle is desirably placed via this check stroke, the caregiver can continue to administer the injection using a delivery stroke. One benefit of performing this dual action in a single stroke is that the needle 202 can remain still. This lessens the risk that the needle might inadvertently shift such that the injection is delivered into an undesired area of the body.

FIG. 11B illustrates an example where the stroke of the lever 230 can include one or more segments 110 and 112 to allow for reversing direction of the plunger 208. It is noted that the length of the segments 110 and 112 can be adjusted as desired and that the illustrated segment lengths are for exemplary purposes only.

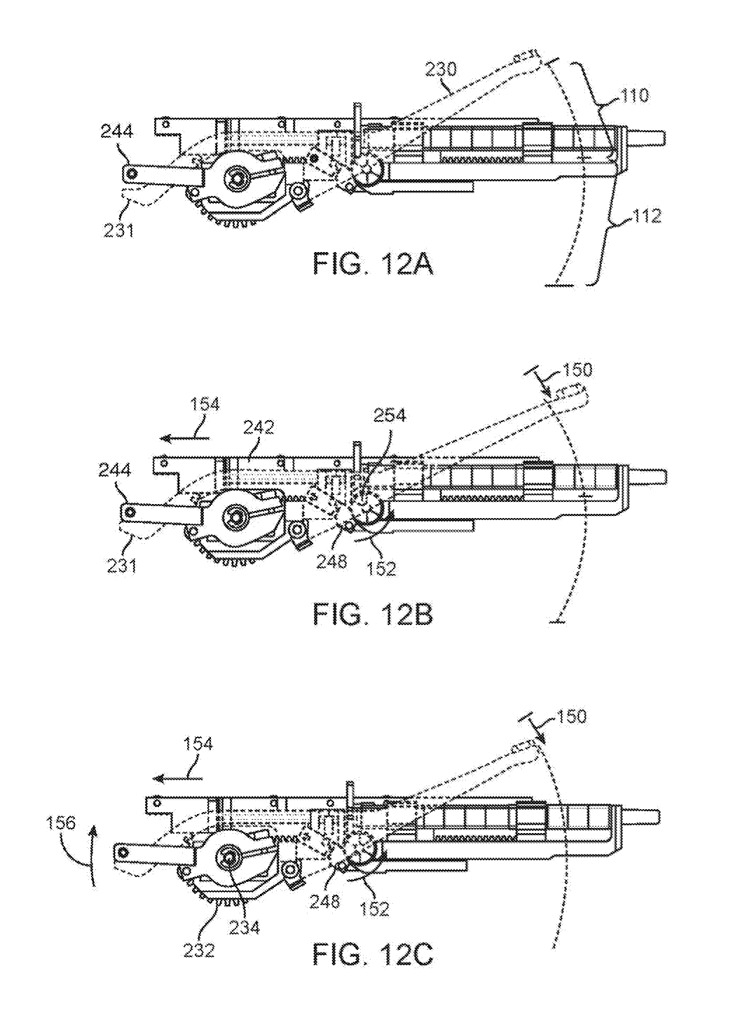

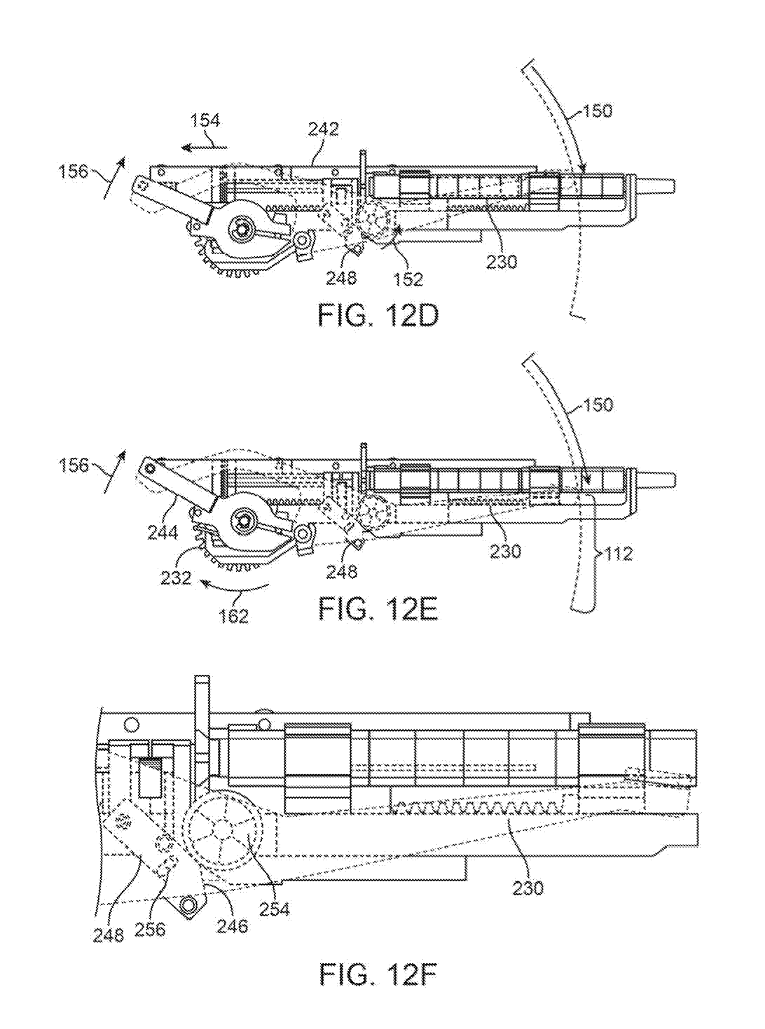

FIGS. 12A to 12E shows an example of the gear drive assembly during movement of the actuator 230 through the first stroke segment 110. FIG. 12A illustrates a rest or initial position of the lever 230. It is noted that the lever as well as other components of the system 100 and/or drive assembly can be spring biased to allow the gears/actuator/etc, to return to an initial position. Continued movement of the lever 230 in direction 150, as shown in FIG. 12B, causes a back drive lever 244 to rotate back drive pulley 236 in direction 152. The back drive pulley 26 is coupled to back drive shaft 236 (shown in FIG. 11A) and rotates back drive gear 236 causing rearward movement of the plunger coupling 242 in direction 154. In the illustrated example, the rear portion 231 of lever 230 has yet to engage main drive lever 244. Once the rear portion 231 of lever 230 engages the main drive lever 248, the main drive lever 248 and rear portion of lever 231 moves in direction 156. However, main drive shaft 234 can employ a one-way clutch such that movement of the lever 244 in direction 156 produces no movement of main gear drive 232. This clutch is necessary since rearward movement of the plunger attachment 242 causes rearward movement of the main rack gear 250.

FIG. 12D illustrates continued movement of lever 230 and rotation of back drive gear 236 and hack drive shaft 238 to continue movement of the plunger coupling 242 in rearward direction 154 FIG. 12E illustrates the condition where movement 150 of lever 230 reaches the second segment 112. In this state, as shown in FIG. 12F, movement 150 of lever 230 into the second segment 112 causes a back drive surface 246 of back drive lever 248 to disengage from back drive pulley 254. In the illustrated example, the slot 256 located in the back drive lever 248 permits movement of the back drive surface 246 away from the hack drive pulley 254. However, any number of configurations (such as a cam/follower design, contoured shape, etc., can be used to prevent movement of the back drive pulley 254). Turning back to FIG. 12E, movement of the lever 230 through segment 112 causes continued movement of the main drive lever 244 in direction 156 causing rotation of the first or main gear 232 in direction 162 resulting in advancement of the plunger coupling 242 as the main gear 232 engages the first rack gear 240 (as shown in FIG. 11A).

Disengagement prevents rearward movement of the plunger fitting so that continued movement of lever 230 through the second segment 112 causes movement of the first gear 232 and first shaft 234 which ultimately causes forward movement of the plunger fitting and plunger to expel the substance located within the reservoir or barrel. Again, the position of lever 230 is provided for illustrative purposes only and the arc length of the first 110 and second 112 segments can be adjusted based on the amount of negative pressure and amount of fluid delivery required. Moreover, gearing ratios can be adjusted as required to obtain desired displacement or movement of the plunger fitting.

In the sequence described above, the first segment 110 corresponds to a check and the second segment corresponds to delivery of the substance. Clearly, if a medical caregiver observes the presence of blood (or any other negative condition), the caregiver can release the actuator 230 and allow the system to revert to the rest or initial position shown in FIG. 12A if the caregiver is attempting to inject a substance and avoid the muscle. Alternatively, the injection system can be used to confirm delivery of a substance into a vessel where the check can be used to confirm placement of the needle within the vessel. The present disclosure includes any number of permutations of use of the system that allows for movement of a plunger in two directions while minimizing movement of the needle and/or with a single stroke of an actuator.

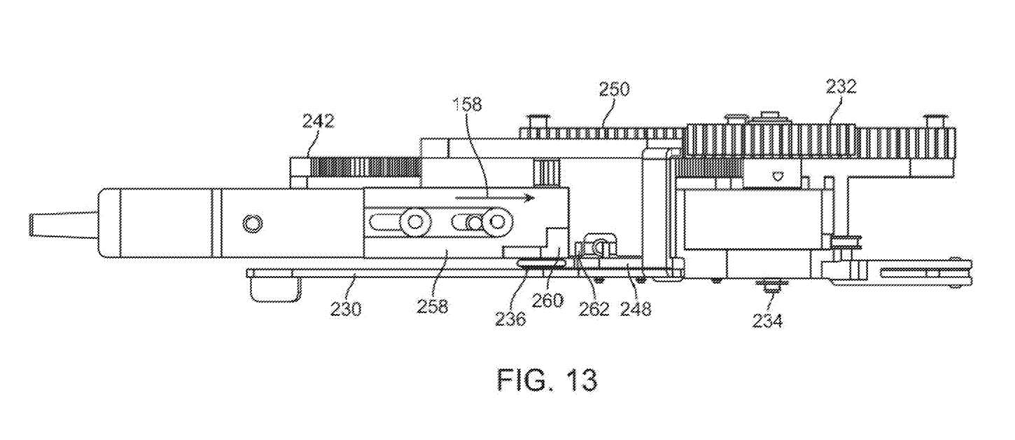

FIG. 13 shows a bottom view of the variation shown in FIG. 10. The illustrated figure shows an optional lockout feature 258 that has one or more surfaces 260 that can engage a portion of the back drive lever 248 (in this example the lockout feature 258 engages one or more pins 262 of the back drive lever 248). In the illustrated example, the lockout feature 258 can be advanced in direction 158 to engage the back drive lever 248 and disengage the device from performing the check feature.

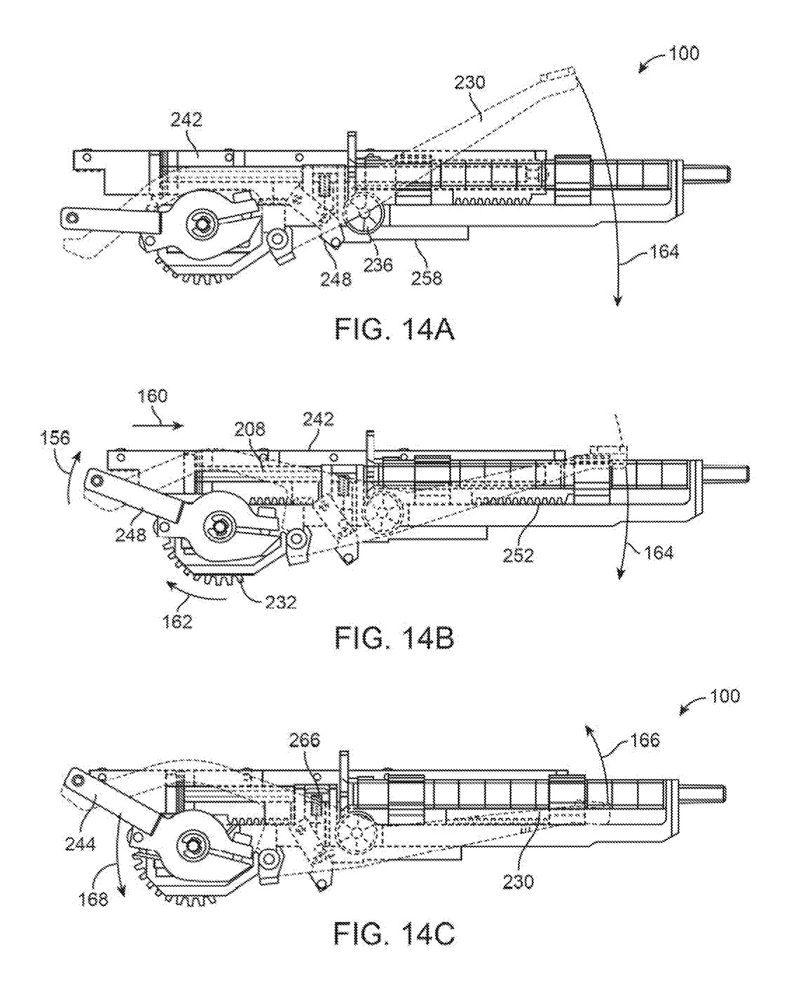

FIGS. 14A to 14C illustrate movement of the actuator 230 to cause advancement of the plunger fitting 242. The examples shown demonstrate the device when the disengagement feature 248 separates the back drive lever 248 from the back drive pulley 236. However, the same principles apply when the back drive arm 248 disengages from the back drive pulley 236 when the actuator 230 enters the second segment 112 of travel as discussed above.

FIG. 14A illustrates the device 100 when the lever 230 is in a rest or initial position and can be moved in direction 164. As shown in FIG. 14B, when lever 230 moves through direction 164, a portion of the actuator engages lever arm 248 and moves lever arm in direction 156. Since there is no counter movement caused by the rear drive pulley 236, main gear 232 moves in direction 162 and engages the main rack gear 252 of the plunger fitting 242 to move the plunger fitting 242 in direction 160. As shown, this causes plunger 208 to move as well. FIG. 14C shows the actuator 230 at the end of a stroke. Again, the position of the actuator 230 relative to the remainder of the device 100 can be varied as needed. Some variations of the invention 100 include a spring reset feature provided by one or more springs 266 that allow the main lever 230 to revert to the initial position shown in FIG. 14A upon removal of an applied force to the lever 230. The spring reset feature also reverts the lever arms 248 244 to their rest positions. In the illustrated example, spring 266 causes movement of actuator 230 in direction 166, where the slot feature can return back drive lever 248 and a second spring (not shown) assists in movement of lever 244 in direction 168.

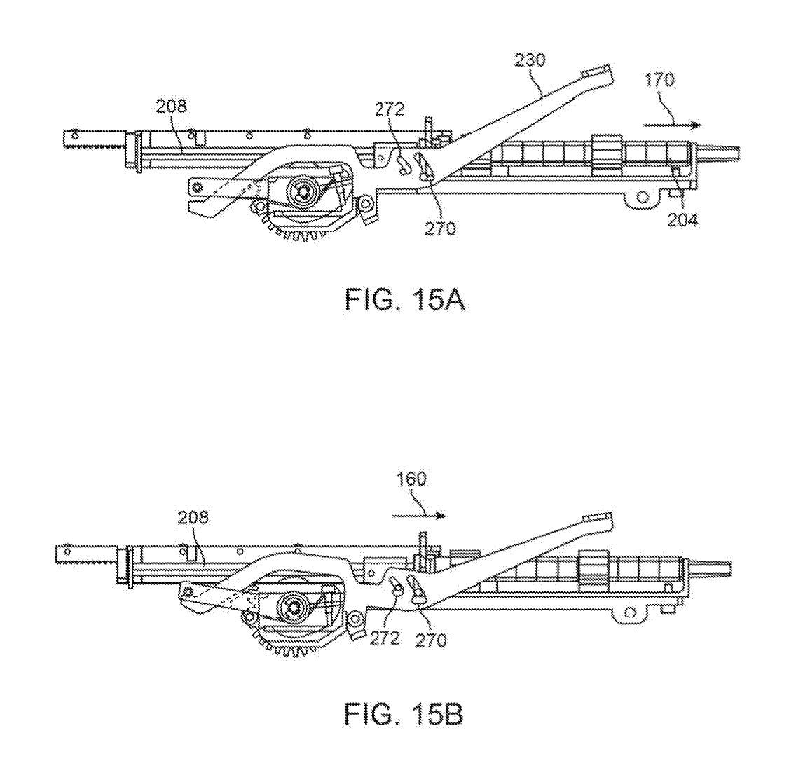

The present disclosure contemplates any number of variations and modification of the devices shown herein that allow for dual movement of a plunger when delivering a substance. For example, FIG. 15A illustrates an alternate design where an actuator 230 moves a barrel 204 in direction 170 while holding the plunger 208 steady where this relative movement creates a negative pressure in the barrel 204 to perform a check function. FIGS. 15A and 15B show one example of a configuration that performs this relative movement where the actuator 230 includes a first and second slots 270 272 that advance the barrel 204 during an initial movement of the lever 230 and then advances the plunger 208 while maintaining the barrel 204 steady so that the plunger advances in direction 160 to dispense the contents of the syringe 204.

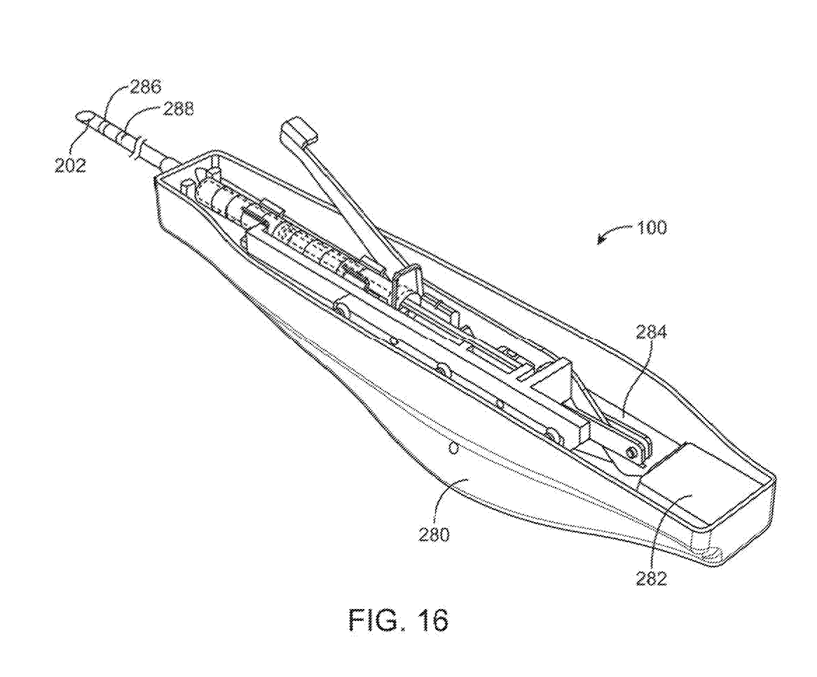

FIG. 16 shows another variation of components for use with the injection systems described herein, as shown, the unit 100 can include any type of outer covering 280 or housing that optionally aids in single handed administration of the device. For purposes of illustration, the housing 280 is shown without a top covering. Furthermore, the housing 280 can include any power supply, electrical contact, memory, or processor (as designated by 282) that can perform sensing through a cannula 202 and/or through any number of electrodes 286. 288 disposed on or in the cannula 202. The circuitry 282 can be coupled to the needle/electrodes 284 using any conventional conductive member 284.

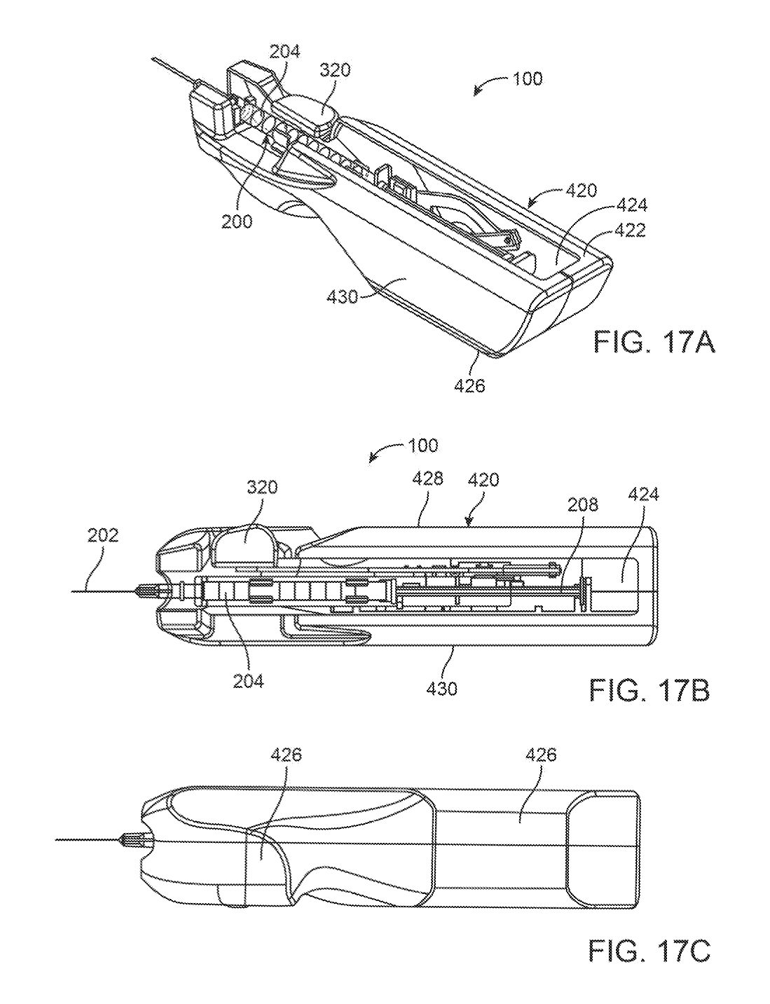

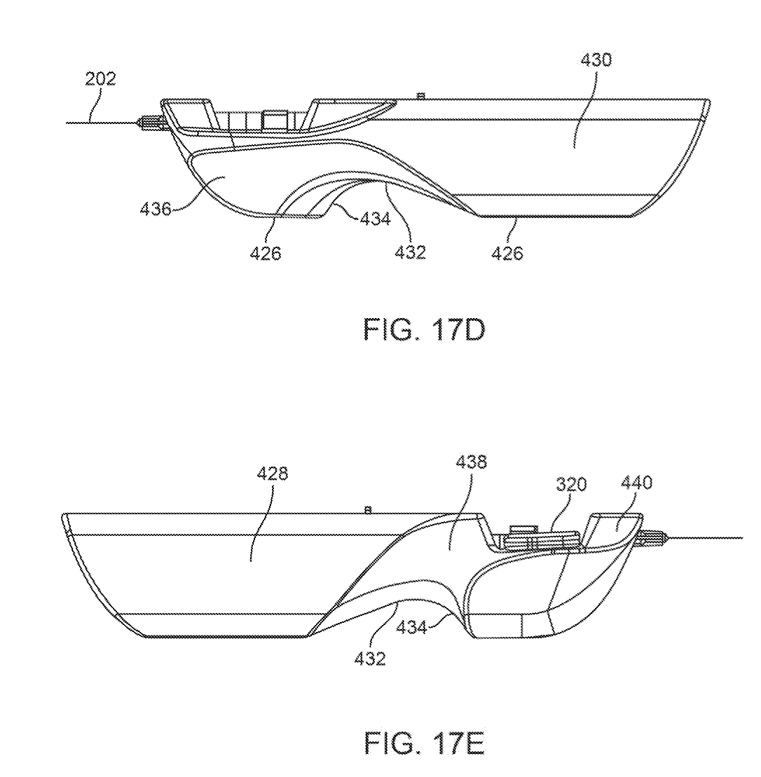

FIG. 17A illustrates a perspective view of another example of a dispensing unit 100 having a modified housing 420 that improves the ability of a user to administer a single-handed positioning and actuation of the unit 100. The illustrated example also shows a plunger 208 slidable relative to a barrel 204 of a syringe 200 that is positioned in the housing 420.

The housing 420 includes one of any number of variations of a drive mechanism and/or gear assembly such as those discussed herein where the drive mechanism and/or gear assembly includes an actuator 320 that moves the drive mechanism as discussed herein to advance a plunger fitting that is coupled to a plunger 208 of the unit (as shown in FIG. 17B).

As discussed below, the housing illustrated in FIG. 17A is designed with a number of features that improve the ability of the medical caregiver to render metered injections.

FIG. 17B illustrates a top view of the unit 100 of FIG. 17A. AS shown, the gear assembly and syringe of the unit can be located within a. window 424 of the housing 420. In the illustrated variation, the window 424 is exposed along a top loading surface 422 that permits loading of the syringe 200 into the gear assembly. In the illustrated variation, the loading surface 422 and window 424 permits sufficient room for positioning the syringe 200 within the housing 420 so that the syringe and plunger can be coupled to the appropriate parts of the mechanism. In the variation shown, the window 424 exposes the drive assembly allowing for insertion of the syringe into the housing in alignment with a longitudinal axis of the housing 420 such that a distal end of the barrel 204 is adjacent to a distal end of the housing and a cannula/needle extends beyond the distal end of the housing. The window 424 also permits a user to engage the plunger of the syringe with a plunger fitting on the gear assembly. Although not shown, the window can be closed by one or more covers.

FIG. 17C illustrates a bottom view of the housing 420 to illustrate a bottom base surface 426 the housing 420 in a stationary and stable position when positioned on an external surface. This base surface 426 allows a user to maintain stability of the housing as the syringe is joined or otherwise coupled to the unit through the window 424 on the loading surface 422.

FIG. 17D illustrates a side view of the housing 420 of FIG. 17A and shows a support side 430 of the housing 420 as well as a plurality of recesses that assist in single handed operation of the device. For example, the housing 420 includes at least a base recess 432 located in the base surface 426. As discussed below, the base recess 432 allows for nesting of a dorsal web of the user's hand therein. The base recess 432 can also include a directional force surface 434 that permits application of a three in a direction of the injection (towards the needle 202) and therefore lessens the grip strength or force required to advance the needle 202 into the target tissue. Without a directional force surface 434 a user must increase a compressive force on the housing via the user's fingers to drive the needle into tissue.

FIG. 17D also shows a support recess 436 located in the support surface 430 where the support recess 436 accommodates a thumb of the user's hand. FIG. 17E illustrates an opposite view of the device showing a lever side 228 with a lever recess 438 extending from the base 426 to a distal end of the body 420. The lever recess 438 allows an index finger or a middle finger of the user contain/control the device with a single hand as the user actuates the lever 320. The lever recess 438 also assists in maintaining the index finger in a relatively straight profile. The body 420 can flintier include a distal or index recess 440 located in the lever side 428 and adjacent to the loading surface 422. The distal/index recess 438 permits nesting of a user's middle finger to balance the body 420 as the user's index finger engages the actuator 320. As shown below, the support recess 436 and the base recess 432 allow nesting of the body 420 in a dorsal web of the user's hand while the distal recess 440 allows the user to maintain a force on the lever side 428 of the body 420.

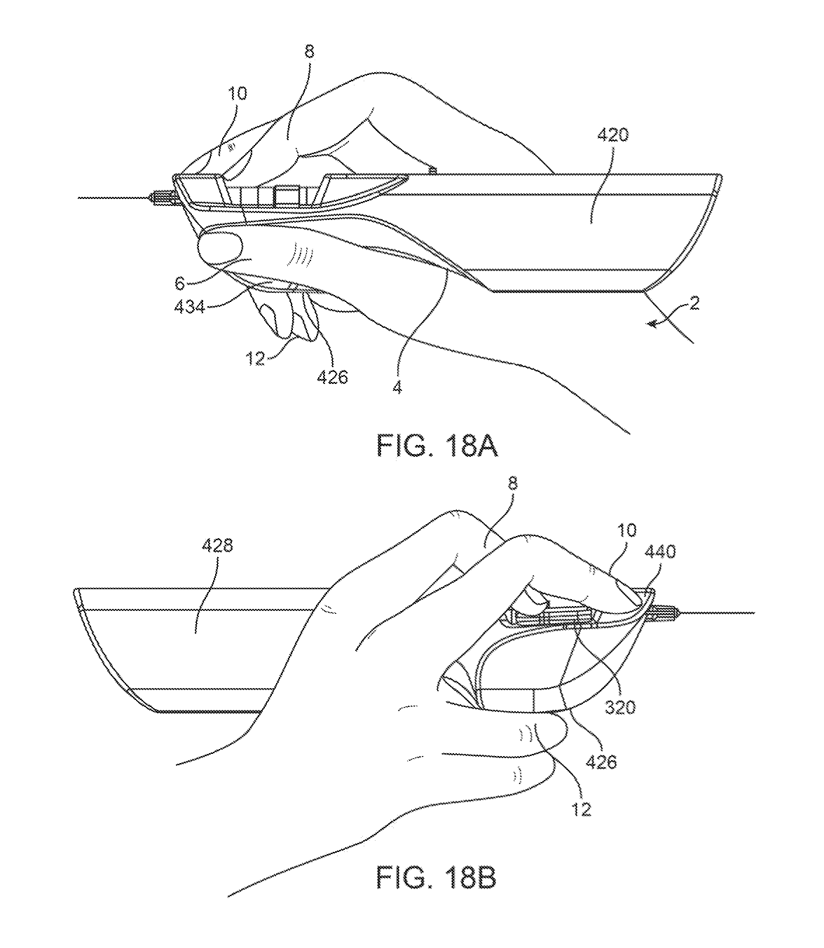

FIGS. 18A and 18B illustrate an example of how the housing 420 of FIG. 17A-17E facilitates one-handed operation of the injection unit. As shown, a user can position the base recess 432 with a web 4 of the user's hand 2 while extending a thumb 6 along the support recess 434. As noted above, the base recess 26 also includes a distal force surface (not shown in FIGS. 18A or 18B). The user's hand (especially the palm or area adjacent to the heel of the thumb) can be used to apply a force in the direction of the needle or a distal force to drive the needle into tissue. This surface reduces the need for the user to apply increased compressive forces to the sides of the housing to drive it forward. FIG. 18B illustrates a lever side 428 of the housing 420 where the middle finger 10 of the user's hand 2 engages the distal recess 440 that is in the lever side and adjacent to the loading surface 422. The distal recess 440 allows the user to apply a stabilizing force on the side of the device opposite to the thumb using the middle finger 10. As shown, the index finger 8 engages the actuator 320 to activate the device. FIG. 18B also illustrates the base surfaces 426 on either side of the base recess 432 provide an added surface area for the user to support the housing either through use of the remaining fingers 12 at the distal portion of the device or at the rear of the device.

As discussed above the configuration of the housing 402 allows for the unit to be held and used with a single hand (e.g., in a manner that resembles holding a pen or where the thumb and first two fingers are oriented in a forward or distal direction). Holding the device using such positioning can enhance controllability and stability of the system during use. Further one handed operation of the system enables its user (e.g., operator or physician) to use his/her free hand to do something else. For example, when using the system to treat wrinkles, one handed operation allows the user's free hand to hold the patient's skin and stretch the skin in the areas of wrinkles, which facilitates the proper placement of the delivered agent with the other hand. The housing configuration shown also provides for surfaces oriented for improved maneuvering to the desired site and improved penetration of the syringe needle into the tissue

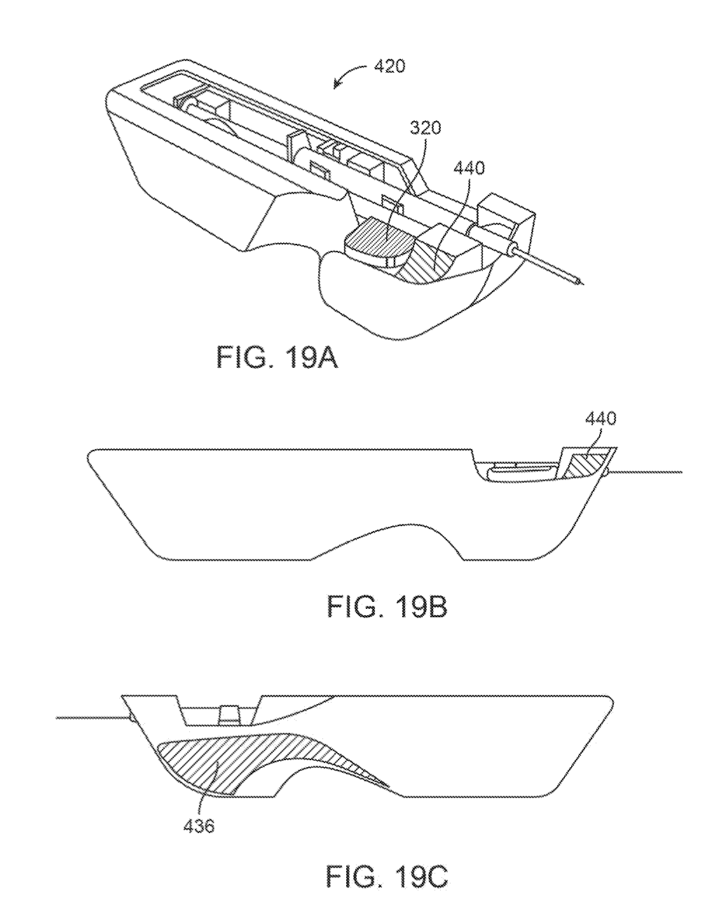

FIGS. 19A to 19C illustrate various views of another variation of a housing 420 where various marked portions of the housing intended for engagement by a user's hand or finger. For example, FIG. 19A illustrates a lever 320 and a distal recess 440 as being marked, either in a visually apparent manner or via a tactile sensation. FIGS. 19B and 19C show an example where a distal recess 440 and support recess 436 are marked and/or identified.

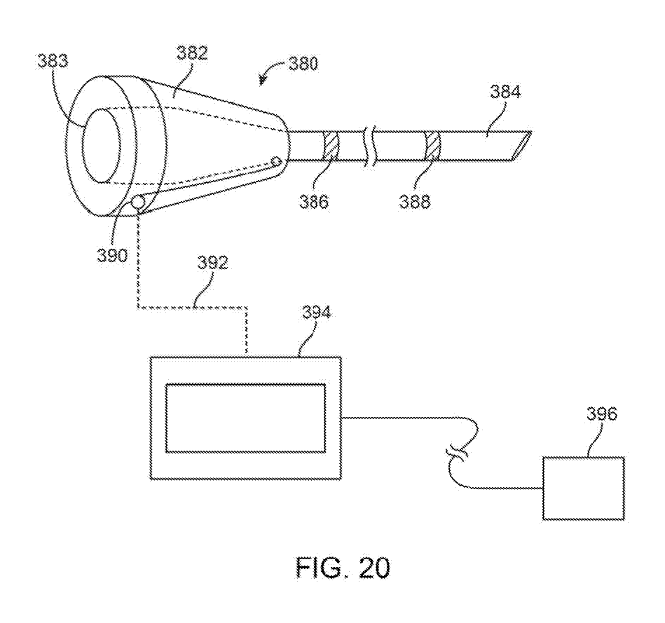

FIG. 20 illustrates another related concept that can be employed with the injection systems described herein or can be employed as a stand-alone improvement to conventional injection devices that gives a caregiver the ability to determine the type of tissue prior to an injection. As shown, a sensing fitting 380 can include a hub 382 that accepts commercially available syringes, fittings, liters, etc. at an opening 383. The hub 382 can include a conductive cannula 384 and/or one or more electrodes 386, 388. The cannula 384 (and/or electrodes) can be coupled to a fitting 390, shown to be on a hub 382 of the device 380 (but could be located in any location including remotely from the fitting 380), where the fitting allows for coupling of the sensing fitting 380 with a power supply/processor/user interface unit/etc. 394. The unit 394 can include any number of user feedback signals such as audible, visual, graphical displays, etc. Once inserted into tissue, the unit 394 can identify whether the caregiver placed the needle into skin, muscle, fat, blood vessels, etc. As noted above, the needle can be used as part of the current path in either a monopolar mode (where the use of a grounding electrode 396 is required, or in a bi-polar mode with either of the two electrodes in combination with the needle or using the two electrodes alone.

The sensing unit 380 can be used when a caregiver must deliver an injection into a certain tissue (e.g., a vessel, skin, fat, muscle, etc.). The conductive needle 384 (and/or electrodes) can provide electrical current to identify the type of tissue through any known modes of identifying such tissue.

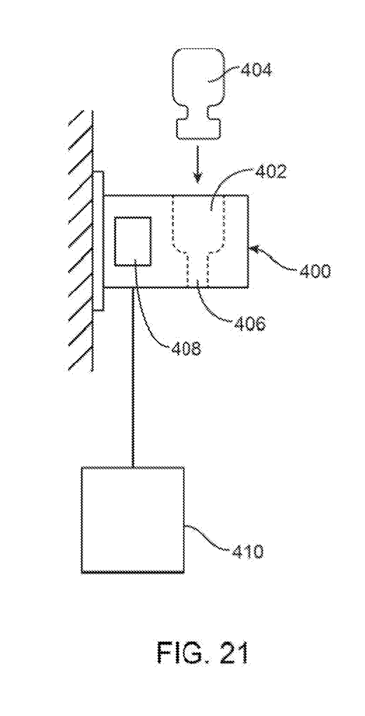

FIG. 21 illustrates yet another aspect for use with the injection systems described herein or with any stand-alone injection system. FIG. 21 shows a holding unit 400 that has an area 402 to accept any type of commonly available vial 402 containing a medicine. The loading unit 400 includes one or more ports 406 for insertion of a needle to draw the substance within a syringe of an injector (not shown). The holding unit 400 is typically coupled to a power supply (either an AC or a DC power supply) that permits the holding unit 400 to maintain the medicine/substance 404 at a desired temperature. Alternatively, the holding unit 400 can include a substance (such as dry-ice, Freon, ice, cold water, hot water, etc.) that can be used to heat or cool the substance 404 without the need for any power supply 410.

Accordingly, the holding unit 400 can include a user interface 408 that provide audible, visual, anchor graphic information regarding the temperature of the substance as well as additional information such as time, batch, type of medicine, etc. The dispensing unit 400 can be affixed to a wall, cart, table, etc. Alternate variations include a portable desktop configuration.

In one example, the dispensing unit shown in FIG. 18 can be used to administer Botox, Xeomin or any similar substance. Such substances are typically reconstituted from a dry lipophilic state. Once reconstituted such substances diminish in potency over time proportional to its temperature. Standard practices include storing reconstituted Botox at a standard temperature of 35 degrees F. The holding unit 400 can prevent having to go to/from freezer/refrigerator when administering. Instead, the holding unit 400 can maintain the vial at the desired temperature allowing for a caregiver to repeatedly access the substance in order to administer injections.

Another added benefit of the bolding unit 400 is that the caregiver is able to load a syringe without holding the vial 404 since the vial is temporarily secured within the holding unit 400.

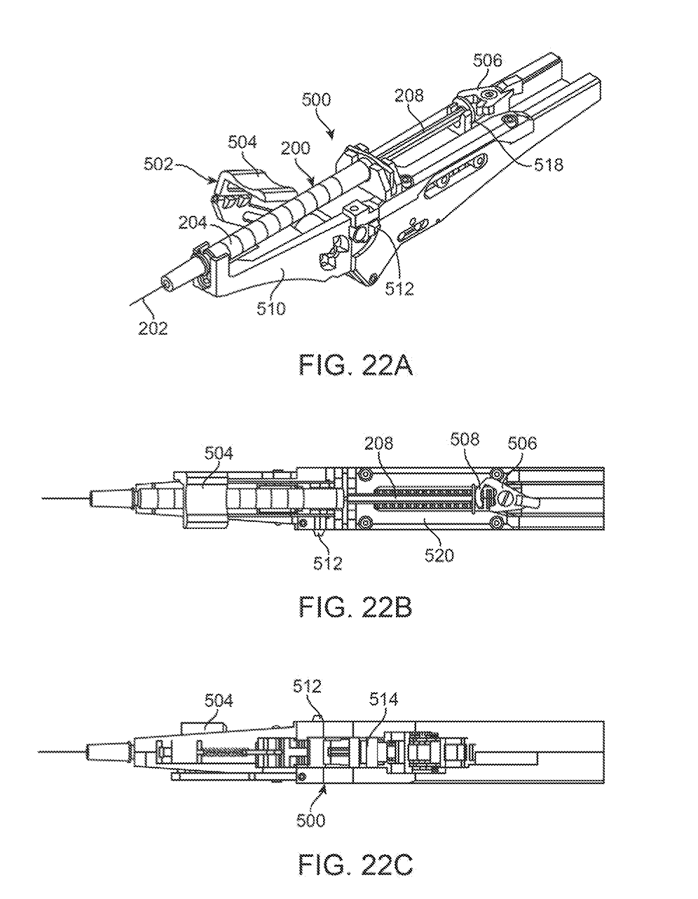

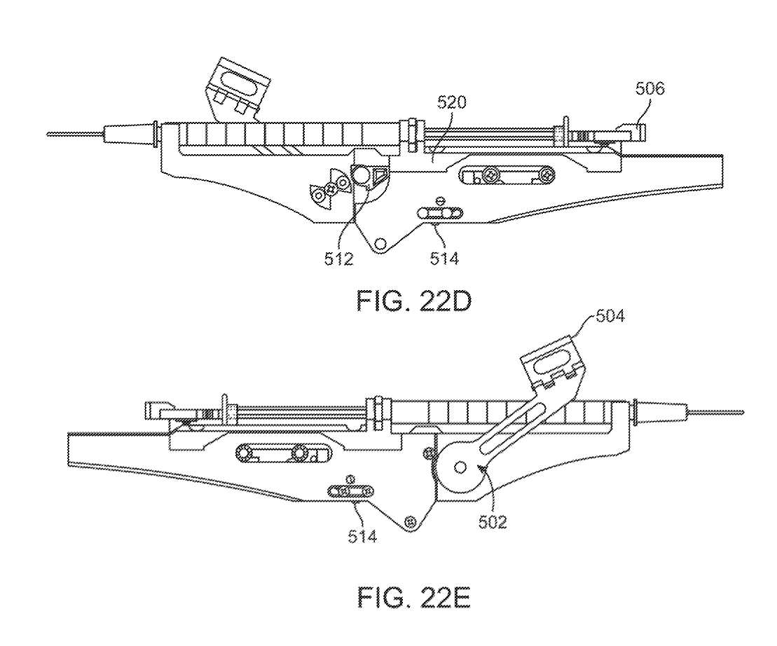

FIGS. 22A to 22E illustrate an additional variation of a dispensing unit 500 for providing injections as discussed above. FIGS. 22A-22E show a perspective view, top view, bottom view, left and right views respectively of a syringe 200 coupled to a body portion 510 of the dispensing unit 500 to secure the barrel 204 as well as the plunger 208 of the syringe 204. The body unit 510 can also include a plunger lock 506 to accommodate a variety of plungers for a variety of syringe designs. In the illustrated variation, the plunger lock 506 is rotatable such that a tensioning component 508 engages the plunger 208. The tensioning, component 508 can deflect when advanced against the plunger 208 to increase a retention force against the plunger as well as to accommodate a variety of dimensional tolerances or shape designs of various plungers.

The dispensing unit 500 can also include an actuator 502 comprising, a moveable portion 504 that can be pivoted or rotated away from the body portion 510 to allow for installation and removal of the syringe 200.

This variation of the dispensing unit 500 also includes a switch 512 to prevent the syringe from drawing blood prior to the injection as addressed above. In this variation, the switch 512 functions as a reverse cancel switch 512 such that it prevents reversal of a plunger fitting 518 when the actuator 502 is advanced. The structure and operation of the reverse cancel switch 512 is discussed below. The unit 500 can also include one or more neutral switches 514 (as shown in FIG. 22C), where the neutral switch allows for disengagement of the drive mechanism 520 from the drive assembly (as discussed below).

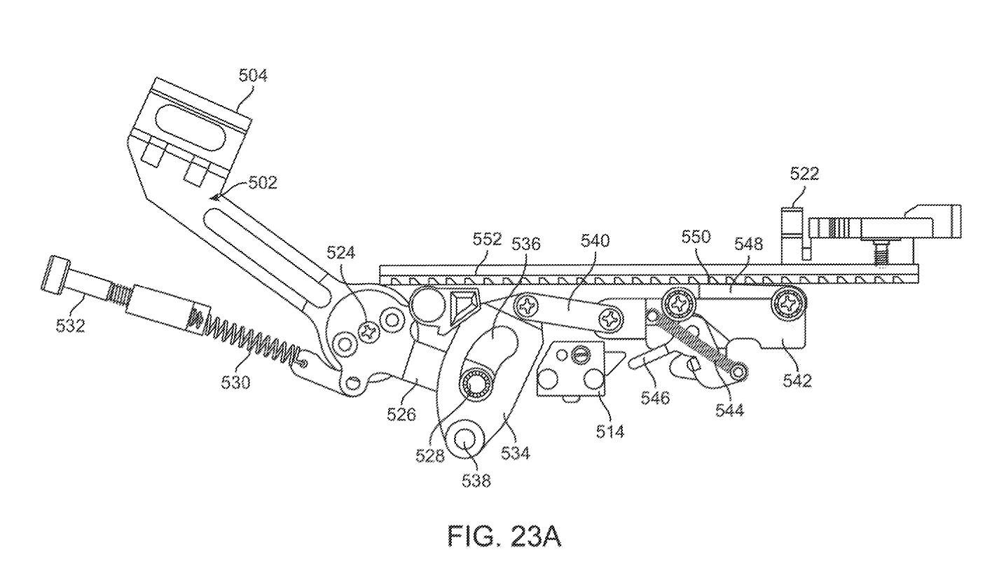

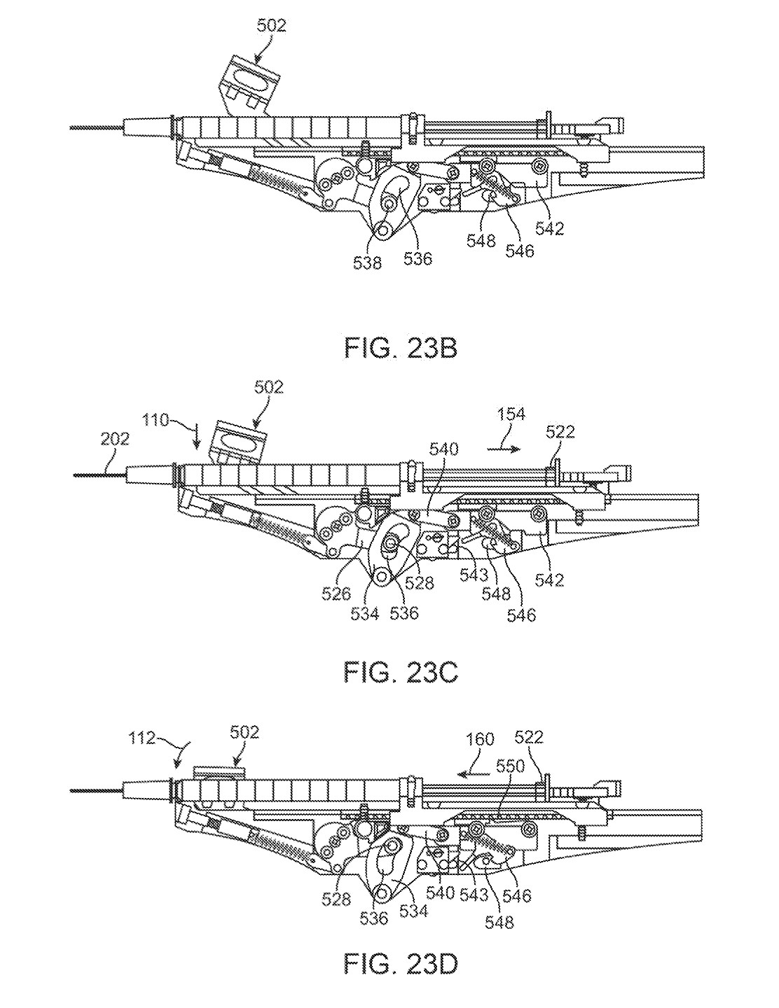

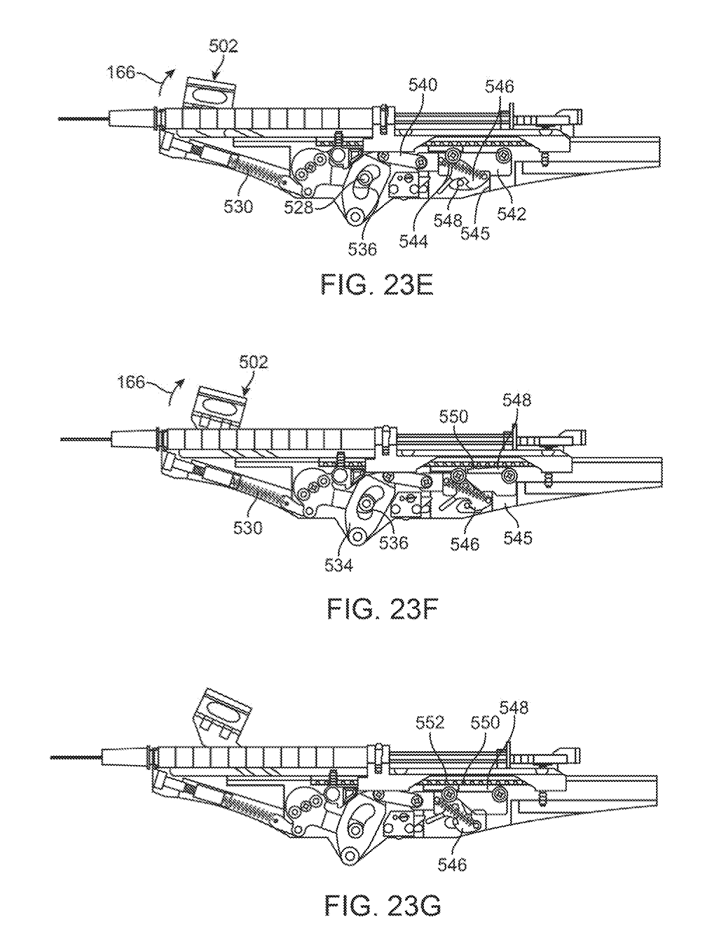

FIG. 23A shows an example of a direct linkage drive assembly for Use with the variation shown in FIGS. 22A-22E. For purposes of illustration, the linkage drive assembly is shown without the housing, syringe, and a number of other components. As illustrated, a lever 502 couples to the drive assembly via an actuator extension 526 which carries a cam follower or bearing 528. The bearing 528 moves in an arc as the lever 502 pivots about a lever pivot point 524. Accordingly, as the lever 502 is driven in a forward or down direction, the cam follower or bearing 528 moves in an upward arc. However, the cam follower or bearing 528 is coupled to a cam or trigger 534. In this variation, the bearing 528 is limited to move within a slot 536 of the trigger 534. The trigger 534 is pivotally coupled to a trigger pivot 538. Therefore, movement of the bearing 528 within the trigger slot 536 causes pivotal movement of the trigger 534 about the trigger pivot 538. The movement of the trigger is illustrated below. The actuator 502 and actuator extension 526 can be tensioned in any manner conventionally used. For example, the actuator 502 can be tensioned by spring 530, which causes actuator to return to a neutral position as shown. Furthermore, the spring 530 can be adjusted using a tensioner screw 532 as shown.

The trigger or cam 534 is coupled to a slide 542 via a trigger linkage 540. Therefore, movement of the trigger 534 (caused by movement of the bearing 528 within the slot 536) pivots trigger 534 causing linkage 540 to move in a linear direction. The linkage 540 is coupled to a rail 552 via a claw 548, slide 542 and hook 546 assembly such that movement of the linkage 540 causes movement of the rail 552 until the hook 546 disengages from the claw 548. In the illustrated example, a portion of the body or any portion of the mechanism can serve as a hook release surface (shown below) that disengages the hook 546 from the claw 548. The claw 548 can be spring biased away from the rail 542 such that when the hook 546 disengages the claw 548, the claw assembly 548 and tooth/teeth 550 move away and disengage from the rail 552.

FIG. 23B illustrates a side view of the drive assembly, coupling the actuator to the drive mechanism where the actuator is at an initial position. FIG. 23C illustrates the actuator 502 moved in an initial back drive stroke 110 causing movement of the cam follower/hearing 528 against the slot 536 of the trigger 534. The shape of the slot 536 causes the trigger 534 to rotate clockwise as the cam follower/bearing 528 moves in an upward direction pushing on a rear side of the slot 536. This causes linkage 540 to move the slide 542 in a rearward direction (relative to the needle 202). Because the hook 546 is engaged with the claw 548, one more teeth 550 of the claw are in a positive or biased engagement with the surface of the rail 552. Therefore, rearward movement of the link 540, slide 542 and claw 548 causes rearward movement 154 of the rail 552 and plunger fitting 522. The resulting movement causes the attached syringe to draw negative pressure in the barrel to test for the presence of the needle 202 in a blood vessel during the initial stroke of the actuator 502. As noted above, the shape of the slot 536 permits this initial backstroke. In the illustrated example, movement of the cam follower/bearing from the position shown in FIG. 23B to the position shown in FIG. 23C applies a force against the rear surface of the slot 536 producing counter clockwise rotation of the trigger 534.

Once the cam follower/bearing 528 reaches the position shown in FIG. 23C further actuator 502 movement 112 (in a downward direction) causes upward movement of the cam follower/bearing 528 towards the position of the cam follower/bearing 528 shown in FIG. 23D. Because the trigger was previously rotated towards a rear of the device, the upward movement the cam follower/bearing 528 now pushes against a far side of the slot 536 to cause counter clockwise rotation of the trigger 534. This counter clockwise rotation of the trigger 534 causes linkage 540 to move the slide 542 in a forward direction (relative to the needle 202). The hook 546 remains engaged with the claw 548 so one more teeth 550 of the claw remain in a positive or biased engagement with the surface of the rail 552. Therefore, forward movement of the link 540, slide 542 and claw 548 causes forward movement 160 of the rail 552 and plunger fitting 522. The resulting movement causes the attached syringe to deliver a metered amount of the substance within the syringe.

FIG. 23D shows the cam follower/bearing 528 as it moves towards the end of the dispensing stroke. In this position, the hook release surface 543 of the body engages the hook 546 (before the end of the end of the dispensing stroke) causing the hook 546 to disengage the claw 548, where the spring biasing of the claw 548 causes disengagement of one or more teeth 550 from the rail 552. FIG. 23E illustrates the movement of the cam follower/bearing 528 as the spring 530 retracts the actuator 502 in direction 166. The retraction of the actuator 502 causes downward movement of the cam follower/bearing 528 which pushes against the rear surface of the slot 536 driving the link 540, slide 542 and claw 548 in a rearward direction. However, because the hook 546 is disengaged from the claw 548 (and where the claw 548 is optionally biased away from the rail), the claw 548 and tooth/teeth 550 move relative/independently of the rail in a rearward direction. This allows incremental movement of the syringe plunger and plunger fitting 552 in a forward direction to continue to dispense substances from the syringe. The rearward movement also causes the hook 546 to engage a hook engaging surface 545 (which can be a portion of the body or any other structure in or on the device). Movement of the hook 546 against the hook engaging surface 545 moves the hook into biased engagement (via spring 544) against the claw 548.

The claw 546 can be released at any time after movement in the reverse direction is completed. This release allows the spring loaded mechanism to become a linear ratchet after the substance is dispensed or at any time of the operators choosing, allowing a full and complete reset of the entire mechanism.

FIG. 23F illustrates continued movement or return 166 of the actuator 502 via the spring 530. The continued return 166 of the actuator 502 maintains movement of the cam follower/bearing 528 in a downward direction where the shape of the slot 536 causes the cam follower/bearing 528 to engage a forward surface of the slot 536 causing counter clockwise rotation of the trigger 534 resulting in forward movement of the link 540, slide 542 and claw 548. As shown in FIG. 23G, this forward movement causes re-engagement of the hook 546 with the claw 548 and re-engages the tooth/teeth 550 of the claw 548 with the slide 552 to position the device in a neutral position but where the plunger and plunger fitting are advanced relative to the position shown in FIG. 23B.

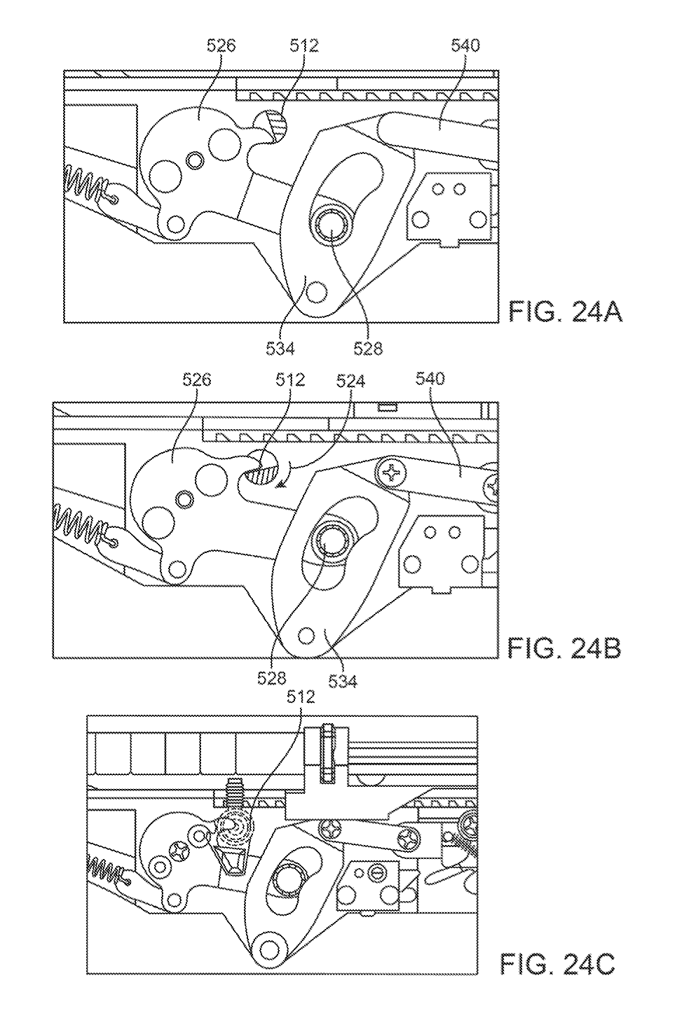

FIGS. 24A to 24C illustrate an optional feature of the device where the reversal action of the drive assembly is temporarily disabled. As discussed above, the device can incorporate a lock 512 that has a first and second positions. In the first position, the lock 512 does not interfere with movement of the drive assembly. In the illustrated partial cross sectional view shown on FIG. 24, the lock 512 is in the position shown in FIG. 23A and allows movement of the device as described in FIGS. 23B to 23G since the lock 512 does not interfere with movement of the actuator extension 526. FIG. 24B shows the lock 512 in a second position as rotated 524 such that a lock surface 512 engages a portion of the actuator extension 526. This interference causes the actuator extension 526 to move the cam follower/bearing 528 to the position shown in FIG. 24B. This position correlates to the position shown in FIG. 23C therefore, at the start of the actuator movement, the cam follower bearing 528 is in a position that will not cause rearward movement of the linkage assembly. Instead, downward movement of the actuator moves the cam follower/heating 528 upwards as described in FIGS. 23C and 23D to drive the plunger fitting to deliver an injection. FIG. 24C illustrates a side view with the lock 512 engaged to prevent rearward movement.

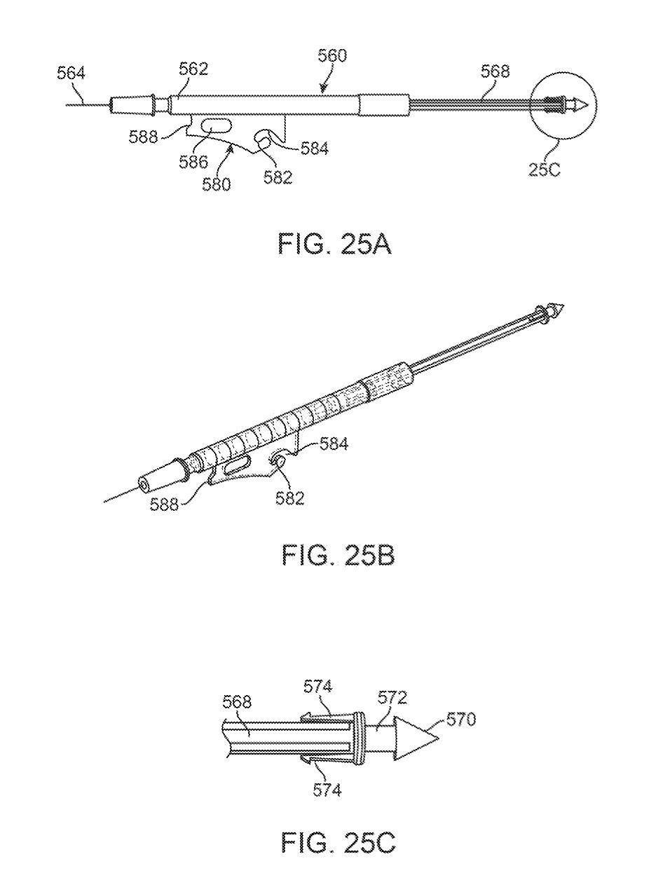

FIGS. 25A to 25C illustrate an improved syringe 560. In the example shown, the syringe 560 includes a retaining member 580 that extends along a length of the syringe body 562 and extends radially away from an axis of the syringe body 562. The retaining member 580 can provide a number of benefits when used in dispensing units such as those described herein. For instance, the retaining member 580 can include one or more retention surfaces 582 that allow engagement of a locking surface (as illustrated below) to releasably lock the retaining member 580 and syringe body 562 to a dispensing unit to prevent movement of the syringe body 562 relative to the dispensing unit.

In the illustrated variation, the retaining member 580 extends along a portion of the syringe body 562 and distributes a force applied by the locking surface along the length of the syringe body to reduce distortion of the syringe body. Such distortion can include axial distortion (i.e., causing a bend in the syringe body away from its central axis), radial distortion (i.e., twisting of the syringe body), distortion affecting cross section of the syringe body (i.e., buckling or compressing of the syringe body), etc.

The retaining member can comprise a flange shape (as shown in the figures) where the flange comprises a planar surface extending parallel to an axis of the syringe body. As shown, variations of the retaining member can extend along at least a half of the length of the syringe body. In alternate variations, the retaining member can extend along a small portion of the syringe body, or alternatively, along the entire length of the syringe body. In most variations, the retaining member is integrally formed with the syringe body. However, variations of the device include one or more retaining members that are joinable and/or detachable from the syringe body.

FIGS. 25A and 25B illustrate a syringe body 562 having retention member 580 in the shape of a planar flange with a plurality of slots 584, 586 where one of the slots 584 includes a retention surface 582. The retention member 580 can comprise any number of shapes with or without slots. In addition, the illustrated variation shows a retention member 580 with a second retention surface 588 at a from of the flange. As shown below, the retention surfaces 582, 588 allow retention of the syringe within a dispensing unit. In the illustrated variation, the flange shaped retaining member comprises at least one slot extending parallel to an axis of the syringe body, where at least a side of the at least one slot that defines the retention surface. As shown, and as discussed below, the slot 584 can comprise an arcuate shape (which mates with an arcuate shape of the locking surface). In addition, the retaining member can include any number of slots.

FIG. 25C illustrates a magnified view of a proximal portion of the syringe 580 of FIG. 25A. As shown, the syringe can include a plunger member 568 with retention tabs 574 that lock into the syringe body 562 (not shown in FIG. 25C) when the plunger 568 is advanced into the syringe body 562. The plunger 568 can further include a retaining feature 572 or groove that locks into a plunger fitting on the dispensing device. The proximal end 570 of the plunger 568 can further optionally include a tapered shape that reduces the potential of a user applying a force to the plunger end 570 in an effort to advance the plunger within the syringe body.

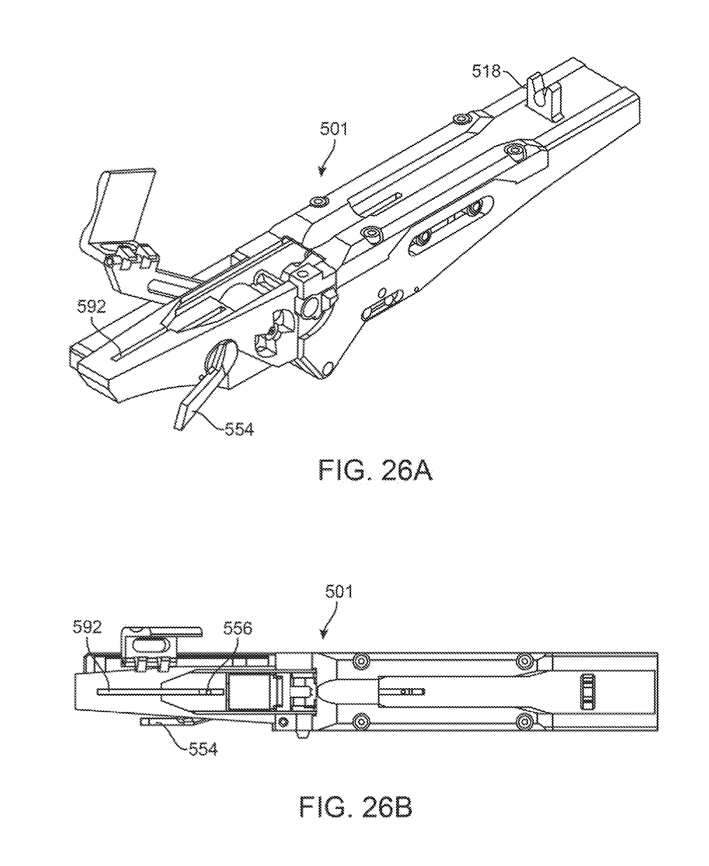

FIGS. 26A and 26B illustrate a variation of a dispensing unit 501 similar to the variations described above with the additional feature of having a locking surface for engaging the retaining member 580 of the syringe 560 shown in FIGS. 25A and 25B. FIG. 26A illustrates a perspective view of the dispensing unit 501. As shown, the dispensing unit can 501 include a slot 592 or other opening through which the retaining member is seated and engages a locking surface (not shown in FIG. 26B), where the locking surface engages and disengages the retaining member through one or more locking actuators 554. The plunger of the syringe removably engages a plunger fitting 518 that is advanced as discussed above.

FIG. 26B illustrates a top view of the dispensing unit 501 of FIG. 26A. As shown, the slot 592 is sized to permit positioning of the retaining member therein such that a locking surface 556 can engage one or more retention surfaces on the retaining member. Variations of the dispensing unit can include a slot 592 that is sized for a friction fit or a sliding fit with the retaining member. Alternatively, the dispensing unit can include an opening rather than a slot. Regardless, the slot/opening can extend fully through or partially within the dispensing unit.

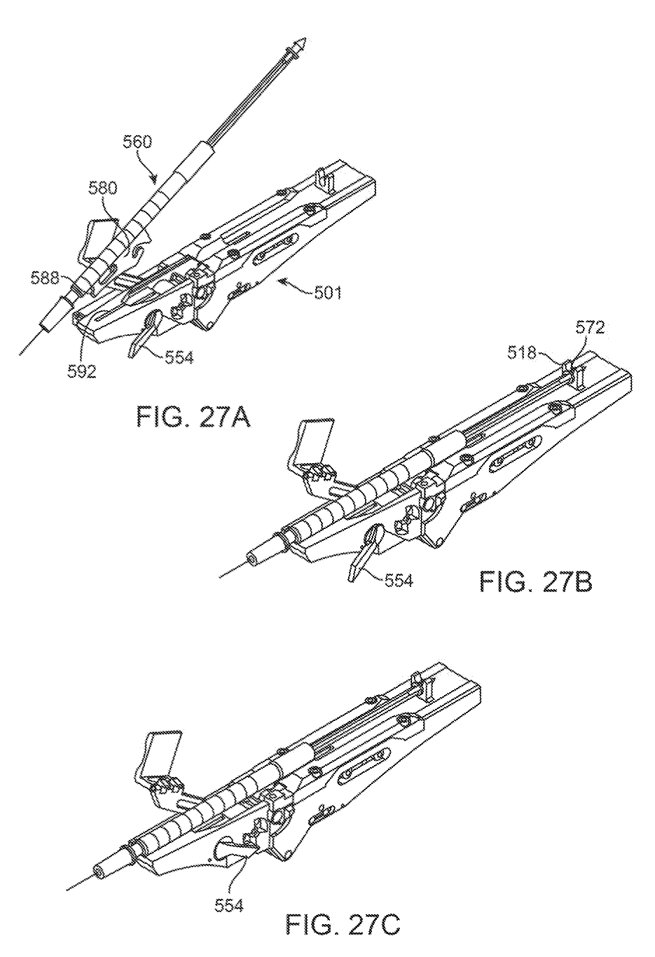

FIGS. 27A to 27C show an example of the syringe 560 of FIG. 25A inserted into the dispensing unit 501 of FIG. 26A. In FIG. 27A, the retaining member 580 of the syringe 560 is aligned with a slot 592 on the dispensing unit 501. The syringe variation 560 includes a retaining surface on a front portion of the retaining member 580, as illustrated below, in this variation the front portion of the syringe 560 and retaining member 580 is inserted first. As shown in FIG. 27B, the retaining groove 572 of the plunger can engage with the plunger fitting 518. At this point, the locking actuator 554, as shown in FIG. 27C can engage the locking surface (not shown in FIG. 27C) into engagement with the retaining surface (not shown in FIG. 27C).

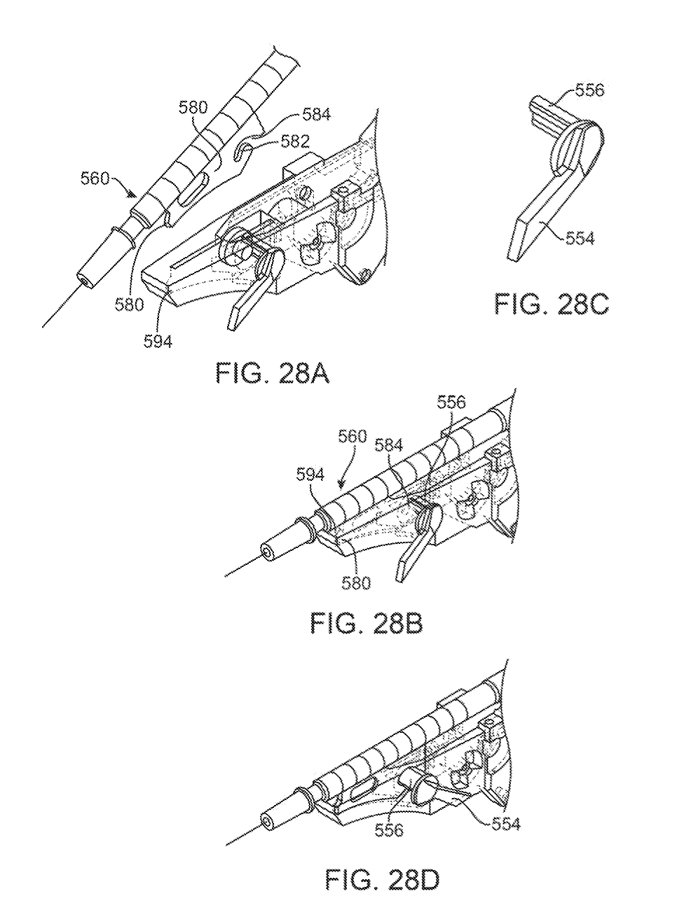

FIGS. 28A-28D illustrate an example of the dispensing unit locking into engagement with the syringe 560. In FIGS. 28A, 28B, and 28D the body of the dispensing unit is shown as transparent/hidden and without the remaining mechanism for purposes of demonstrating the engagement of the locking surface 556 with the retaining surface 582. FIG. 28A illustrates the syringe 560 and dispensing unit in a position similar to that of FIG. 27A as shown above. As illustrated the forward retaining surface 580 is inserted into the dispensing unit first, so that it can be nested within a mating locking surface 594 at the front of the cavity/slot. Alternate variations of the device are not limited to the number of locking surfaces.

FIG. 28B illustrates the front locking surface 580 of the retaining member 580 engaged with the front locking surface 594. As noted above, this variation of the dispensing unit includes a stationary locking surface 594 as well as a moveable locking surface 556. As shown in FIG. 28C, the moveable locking surface 556 can be coupled to a lever or actuator 554. The variation of the locking surface shown in FIG. 28C includes a curved locking surface 556 that matches a curved profile of the slot 584 in the retaining member that forms the locking surface 582. As shown in FIG. 28B, upon engagement of the syringe 560 with the dispensing unit, a portion of the locking surface 556 enters the slot 584. At this point, the lever 554 can be rotated to engage the locking surface 556 within the slot 584 of the retaining member 580. In variations of the device, engagement of the retaining member 556 within the slot 584 and against the rear retaining surface 582 urges the front retaining surface 580 against the stationary locking surface 594.

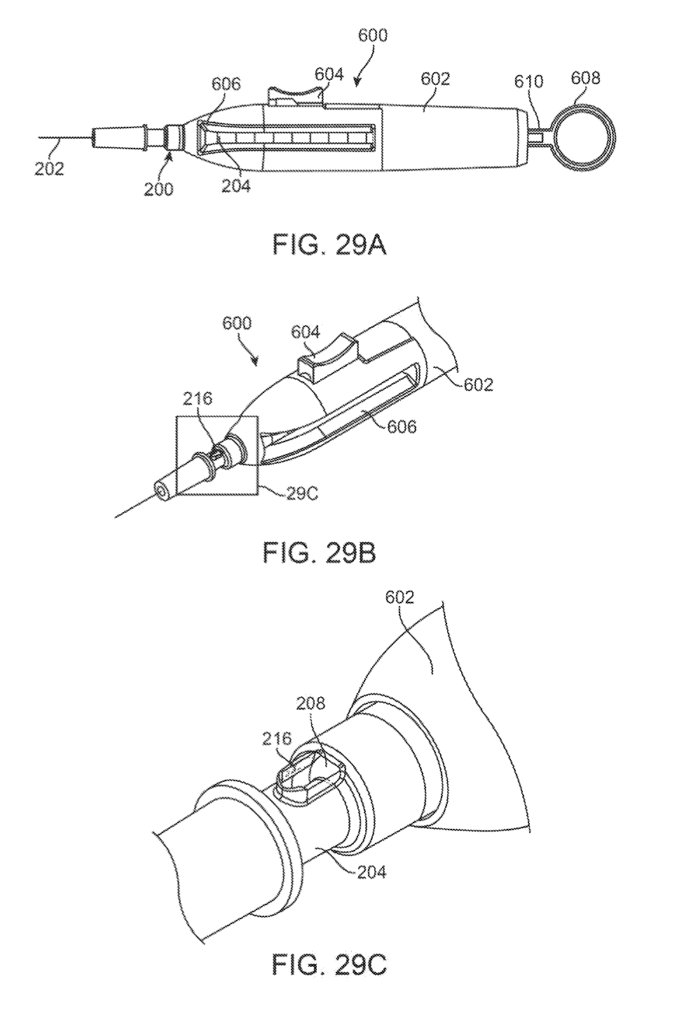

FIGS. 29A to 29C show another variation of a dispensing unit 600, FIG. 29A shows a variation of a dispensing unit that includes a body 602 having a moveable actuator 604 that can dispense a pre-determined amount of a substance within a barrel 204 of a syringe 200 located therein. The body 602 can also include a window 606 that permits an operator to monitor the contents/level of substance within the syringe body 204. As illustrated, the syringe 200 can include a needle 202 coupled thereto or may optionally have a needle integrally formed with the syringe 200.

FIG. 29B illustrates the dispensing unit 600 of FIG. 29A but with an improved syringe 200, which includes a transparent or translucent flash window 216. The flash window 216 is provided to allow an operator to check for fluids within the syringe body 204. For example, in those variations where an operator inserts the needle 202 into a blood vessel, there may be a need to check for blood flashback into the syringe to ensure that the needle is within a vessel. The presence of the flash window 216 allows an operator to check for blood flashback. The window 216 can be located on any portion of the syringe 200 and can optionally provide a magnified view through selective curvature or selection of the flash window 216. FIG. 29C illustrates a magnified view of the section 29C in FIG. 29B. As shown, the flash window 216 allows a user to visually inspect the interior of the syringe 200, in the illustrated variation, a plunger 208 of the syringe is visible. In additional variations, the surfaces within the syringe and adjacent to the flash window 216 can be colored to improve visual detection of blood or fluids. I.e., the surfaces of the interior of the syringe or syringe body can be bright, reflective, or any color/visual indicator to assist in observing fluids within the syringe. The syringe of FIG. 29B with flash window 216 can be used with any variation of dispensing unit described herein and can also be combined with the syringe of FIGS. 25A-25C.

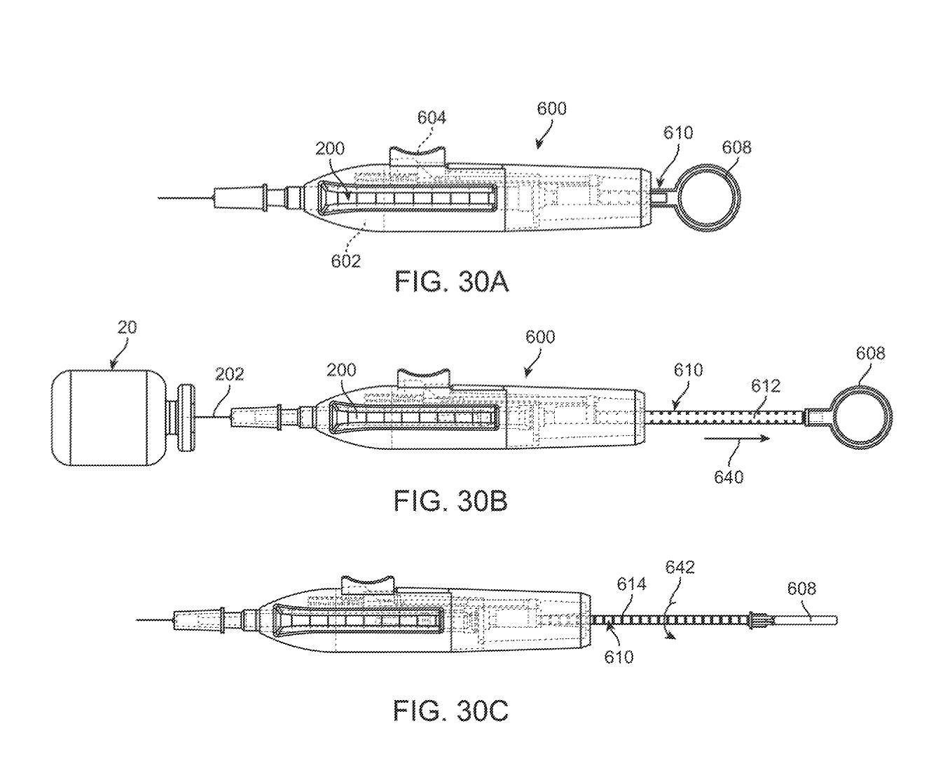

FIGS. 30A to 30C illustrate a filling process of the dispensing unit 600. For purposes of illustration, the body 602 and actuator 604 are shown as transparent/phantom views. FIG. 30A illustrates an initial position of the dispensing unit 600 with the plunger 610 of the syringe 200 in a storage position. FIG. 30B represents a state where a user positions the dispensing unit 600 so that a needle 202 is in fluid communication with a substance to be injected (in the illustrated example, the needle 202 advances into a vial 20 containing the substance to be injected). Variations of the device include fluidly coupling the syringe body 208 with a substance without using a needle. Once fluidly coupled, the user retracts the plunger 610 as shown by arrow 640. The plunger 610 can optionally include a pull ring structure 608 that assists the operator in identifying the orientation of a smooth surface 612 and a slotted surface 614 along the plunger 610. The slotted surface and/or smooth surface can be flat, curved, or angled. Alternate variations of the device include any structure (aside from a ring 608) that assists in pulling back of the plunger 610 and/or identification of the orientation of the smooth surface 612 or slotted surface 614 of the plunger 610. The orientation of the plunger 610 allows retraction or advancement of the plunger 610 and is discussed below.

Withdrawing the plunger 610 causes the substances to be drawn within the syringe 200. In order to deliver the substance, the operator rotates the plunger 610, as shown in FIG. 30C and demonstrated by arrow 642. Rotation of the plunger 610 causes the slotted surface 614 to engage surfaces on a collet (disclosed below) that allow advancement of the plunger 610 and delivery of the substances from within the syringe 200.

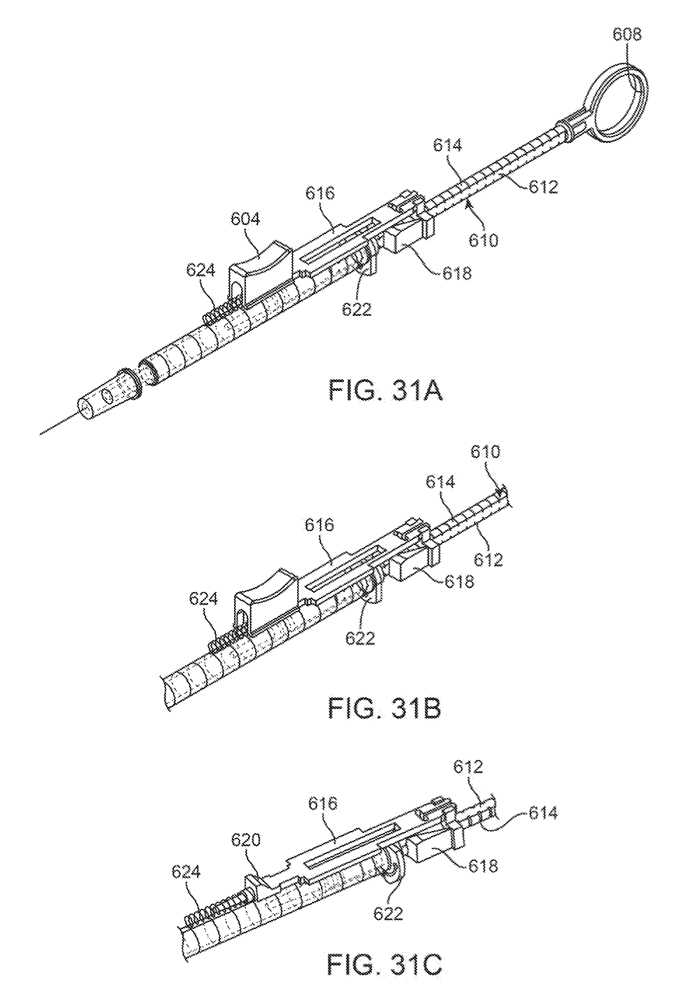

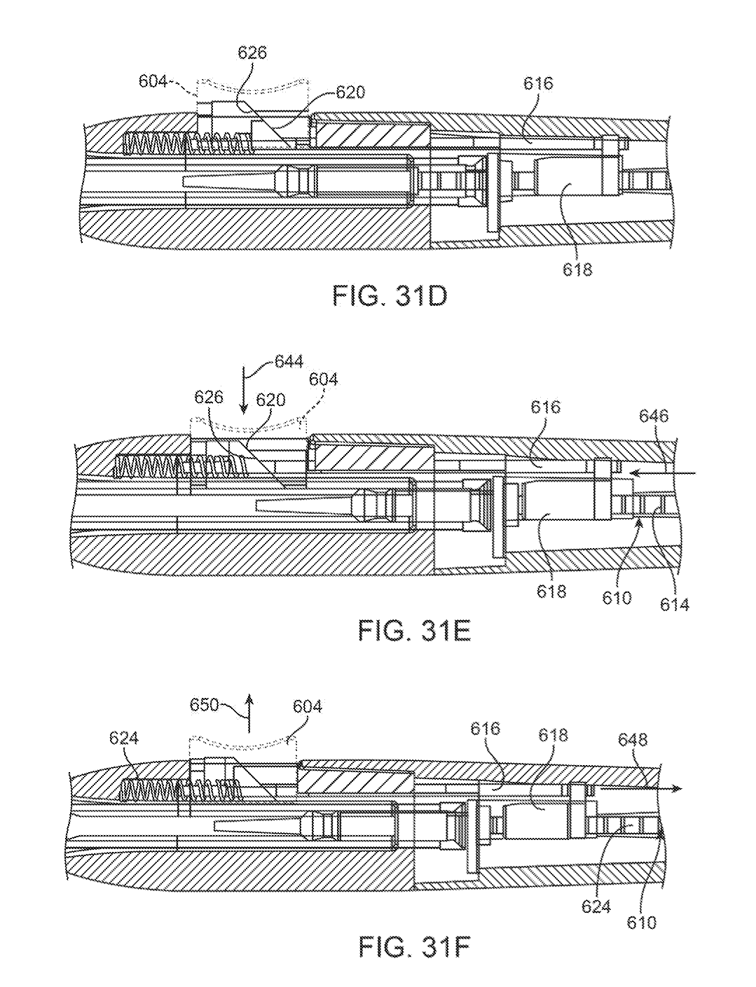

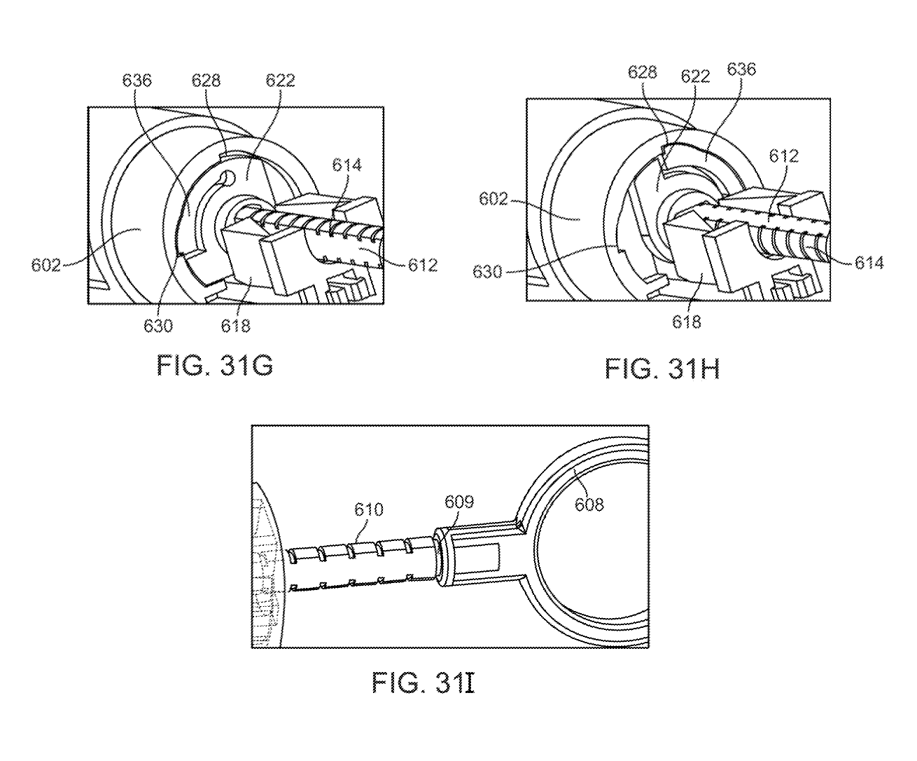

FIGS. 31A-31C illustrate the dispensing unit 600 of FIG. 30A with the body 602 of the unit 600 hidden to better illustrate a drive mechanism of the unit 600. As illustrated, an actuator or button 604 is mechanically coupled to the plunger shall 610 by a cam slider 616 and a collet 618. FIG. 31B illustrates a condition of the drive mechanism where the plunger 610 is capable of being withdrawn as illustrated in FIG. 30B. In this configuration, the smooth side 612 of the plunger 610 engages sharp edges of the collet 618. The smooth side of the plunger allow the collet 618 to slide against the plunger 610. FIG. 31C illustrates the state of the device where the plunger 610 rotates, as shown in FIG. 30C. Rotation of the plunger 610 engages the slotted surface 614 of the plunger 610 with the sharp edges of the collet 618. Therefore, in this position, movement of the collet 618 will cause movement of the plunger 610. FIG. 31C depicts the mechanism of FIGS. 31A and 31B with the actuator 604 hidden to better illustrate a cam surface 620 of the cam slider 616.

FIGS. 31A to 31C also illustrate an optional locking mechanism 622. In use, the locking mechanism 622 prevents reverse rotation of the plunger 610 after the slotted surface 614 engages the collet 622. Alternatively, the locking mechanism 622 can provide a safety mechanism that must be disengaged prior to rotation of the plunger 610.

FIGS. 31D to 31F illustrate an example of the drive mechanism of the dispensing unit of FIG. 30A. As shown, the cam surface 620 of the cam slider 616 engages a similarly sloped surface 626 on the interior of the actuator 604. When the actuator 604 is depressed, as shown by arrow 644 in FIG. 31E, the interior surface 626 of the actuator 604 moves against the cam surface 620 to drive the earn slider 616 in direction 646. The collet 618, being attached to the cam slider 616 and engaged with the slotted surface 614 of the plunger 610, moves with the cam slider 616 to advance the plunger 610. The amount of plunger 610 movement for a full depression of the actuator 604 can be configured to correspond with a specific dosage through the angle of the cam surface 620. Therefore, variations of the device permit delivery of a single desired dose as the result of a single stroke of the actuator 604. FIG. 31F illustrates the return stroke 650 of the actuator 604. As shown, the cam slider 616 is spring biased by spring element 624 to return in direction 648. The tapered shape of the collet 618 (as shown in FIG. 31C), permits the collet 618 to disengage from the slotted surface 624. This disengagement permits the cam slider 616 and collet 618 to move in direction 648 while plunger 610 remains stationary. Therefore, a subsequent movement of the actuator 604 again dispenses a similar amount of the substance located within the syringe body.

FIGS. 31G and 31H show an additional variation of the dispensing unit 600 of FIG. 30A for locking the plunger between a storage position, where a user can retract the plunger as shown in FIG. 30B above, and a ready position, as shown in FIG. 30B.

FIG. 31G illustrates a view along the plunger end of the device with a portion of the body 602 hidden to illustrate a locking mechanism 622 having a locking arm 636 engaging a recess 630 within the body 602 or within a component located within the body. In this variation, the locking mechanism 622 is coupled to the plunger so that rotation of the plunger causes rotation of the locking mechanism 622. Resistance of the locking arm 636 against the first recess 630 provides a threshold force that must be overcome to rotate the plunger. As shown, in FIG. 31G, the smooth side 612 of the plunger engages the collet 618 when the locking arm 636 is engaged within the first slot 630.

FIG. 31H shows the state of the dispensing unit when the plunger is rotated such that the slotted surface 614 engages the collet 618. Rotation of the plunger also rotates the locking mechanism 622 causing the locking arm 636 to deflect as it rotates from the initial recess 630 and engage a second recess 628. The second recess 628 can be shaped to engage the locking arm 636 in such a manner that prevents reversal of the locking mechanism 622 and therefore prevents reversal of the plunger. Accordingly, this one-way detent configuration permits prevents rotation of the plunger to position the smooth side 612 of the plunger against the collet 618. Therefore, when in the configuration shown in FIG. 31H, the dispensing unit is ready for dispensing but is locked to prevent rotation of the plunger and therefore withdrawal of the plunger.

FIG. 31I illustrates an additional optional feature in which the plunger 610 includes a high-stress or break-away section 609 between the plunger shaft 610 and the ring structure 608 (or any other structure that facilitates withdrawal and/or rotation of the plunger 610). The high-stress or break-away section 609 can he engineered such that it breaks upon the application of a sufficient rotational force or torque applied to the ring in an attempt to overcome the one-way detent of the locking mechanism 622.

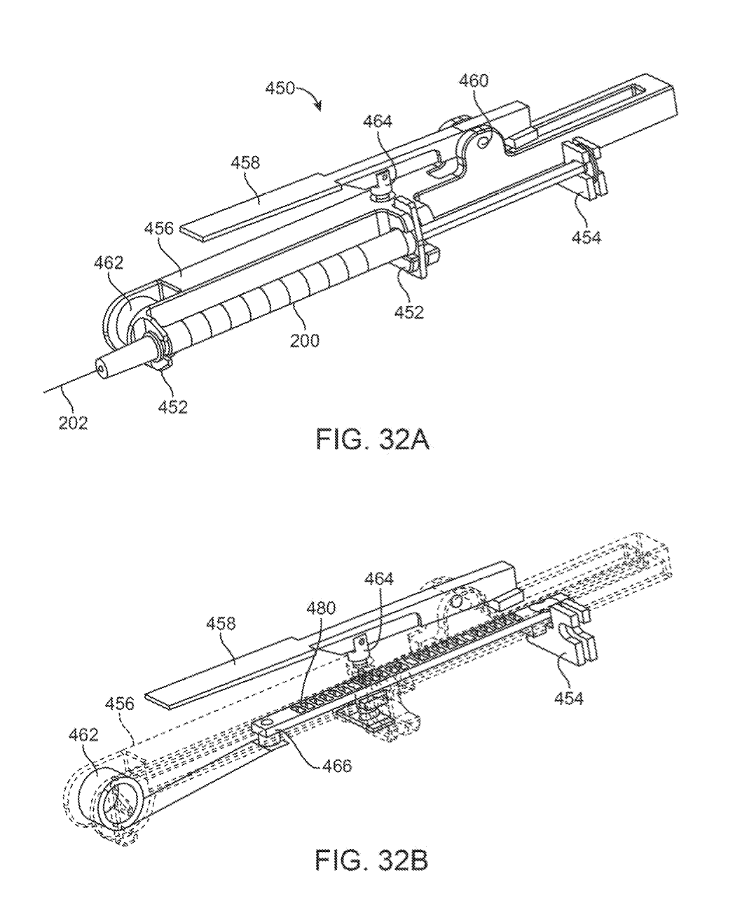

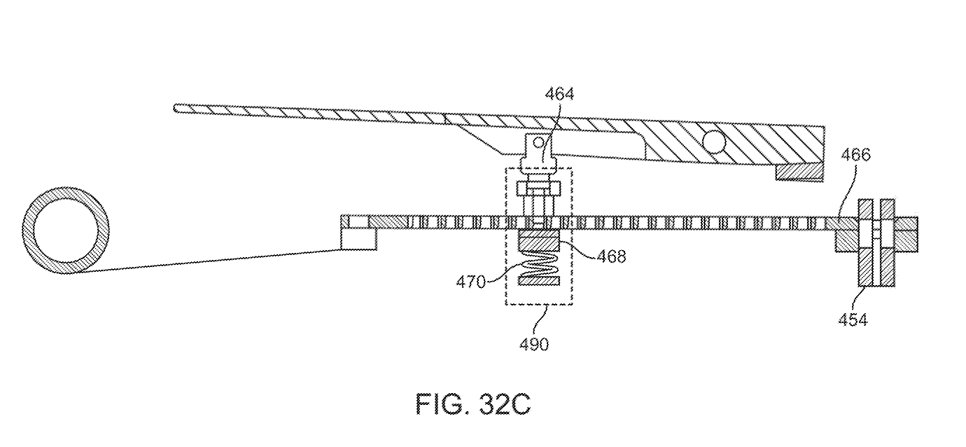

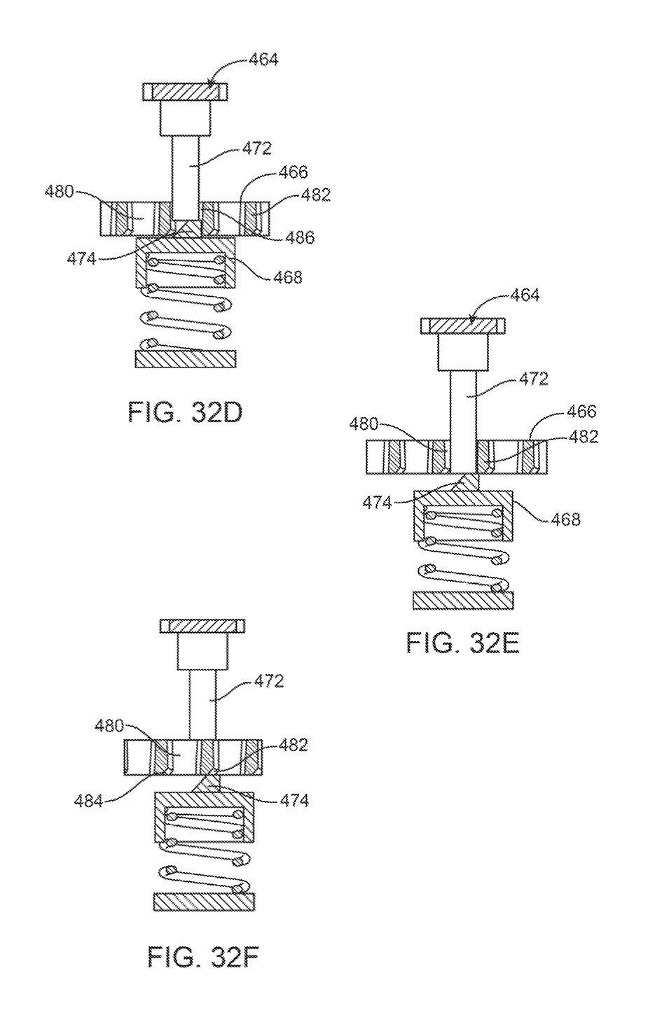

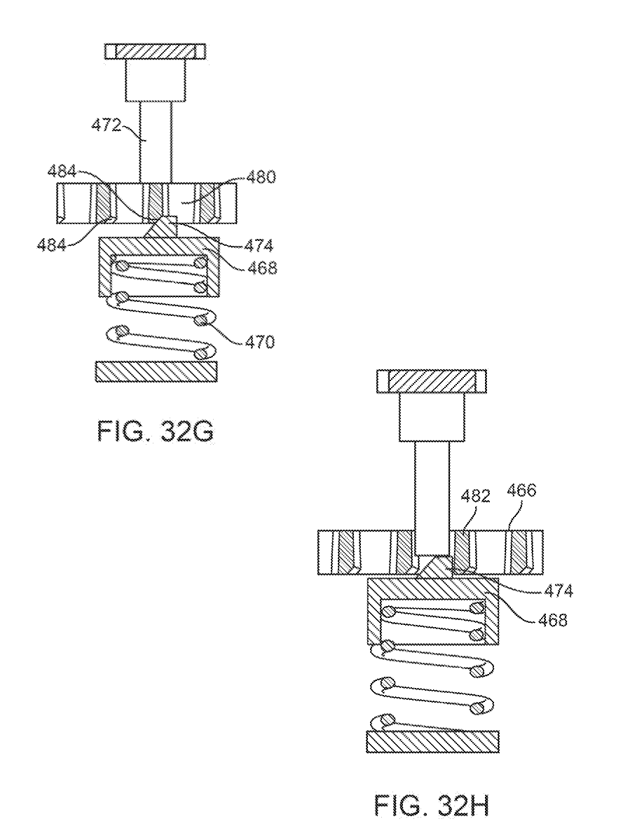

FIG. 32A shows another variation of a dispensing unit 450 that employs a constant force spring/clock spring 462. As shown, the dispensing unit 450 includes a body portion 456 having, syringe retainers 452 as well as a plunger fitting 454. The dispensing unit 450 also includes an actuator 458 pivotally coupled at location 460 to the body 456. The actuator can be spring biased away from the body 456 of the unit 450. The actuator 458 is also coupled to an ejector assembly 464.

FIG. 32B illustrates the dispensing unit 450 of FIG. 32A with the syringe 200 removed and the body portion 456 hidden for illustration of the drive assembly. As shown, the spring 462 is coupled to a slide rail 466. To operate the unit 450 depression of the actuator 458 causes ejector assembly 464 to displace a catch pin (not illustrated in FIG. 32B) causing the constant force spring 462 to draw the slide rail 466 and plunger fitting 454 in a forward direction to cause relative movement between the plunger fitting 454 and the syringe fitting 452.

FIG. 32C illustrates a side view of the dispensing unit of FIG. 32B with the body hidden to better illustrate the movement of the ejector assembly 464 to cause advancement of the slide rail 466. As shown, the ejector assembly 464 engages a catch pin 468, which is spring biased by spring 470 within an opening 480 in the slide rail 466 to prevent movement of the slide rail 466. FIGS. 32D to 32H illustrate a magnified view 490 of FIG. 32C to illustrate advancement of the slide rail 466.

FIG. 32D illustrates an initial position where an ejector pin 472 is positioned within an opening 480 of the slide rail 466 and against a protrusion 474 of the catch pin 468 to prevent movement of the slide rail 466. As shown, the sizing of the pin 472 relative to the opening 480 allows for a gap 486 between an edge 482 of the slide rail 466 and the pin 472.

FIG. 32E illustrates a condition where the actuator (not illustrated in FIG. 32E) depresses the ejector pin assembly 464 causing movement of the protrusion 474 of the catch pin 468 out of the opening 480. In this condition, the slide rail 466 is pulled so that the edge 482 of the slide rail 466 moves against the pin 472. This causes the edge 482 of the slide rail 466 to restrain the protrusion 474 and compress the spring 470 as shown in FIG. 32F.

FIG. 32G illustrates the condition where the actuator (not illustrated in FIG. 32G) is released causing retraction of the ejector pin 472 out of the opening 480, which causes the constant force spring (not shown in FIG. 32G) to move the slide rail 466 and plunger fining resulting in ejection of a substance from within the syringe body. As the slide rail advances, a tapered edge 482 of the opening 480 allows the protrusion 474 of the catch pin 468 to re-enter an adjacent opening 480 prior to the pin 472. The slide rail 466 continues to advance while the spring 470 drives the catch pin 468 into the opening 480 until the edge 480 of the slide rail 466 advances against the protrusion 474 of the catch pin 468.

Any feature described in any one embodiment described herein can be combined with any other feature of any other embodiment whether preferred or not.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto.

Although the present methods and devices have been described in terms of the embodiments above, numerous modifications and/or additions to the above-described preferred embodiments would be readily apparent to one skilled in the art. It is intended that the scope of the present inventions extend to all such modifications and/or additions and that the scope of the present inventions is limited solely by the claims of the invention.

1. A syringe for use with a dispensing unit, the dispensing unit haying a locking surface being moveable for releasably locking the syringe therewith, the syringe comprising:
a syringe body comprising a reservoir located therein;
a retaining member comprising a planar surface extending along a length of the syringe body and extending away from the syringe body, a retention surface formed by a slot extending entirely through the planar surface wherein engagement of the locking surface against the retention surface releasably locks the retaining member and syringe body to the dispensing unit preventing movement of the syringe body relative to the dispensing unit, and where the retaining member distributes a force applied by the locking surface along the length of the syringe body to reduce distortion of the syringe body.

2. The syringe of claim 1, where a portion of the retention surface is parallel to an axis of the syringe body.

3. The syringe of claim 1, where the retaining member comprises a flange shape.

4. (canceled)

5. The syringe of claim 3, where the slot comprises an arcuate shape.

6. The syringe of claim 3, where retaining member comprises a plurality of slots.

7. The syringe of claim 1, where the retaining member extends along, at least a half of the length of the syringe body.

8. The syringe of claim 1, where the retaining member is detachable from the syringe body.

9. The syringe of claim 8, where the retaining member is integrally from with the syringe body and detached through a weakened area.

10. The syringe of claim 1, further comprising a luer fitting at a distal end of the syringe body.

11. The syringe of claim 1, further comprising a plunger member slidably positioned within the syringe body.

12. The syringe of claim 9, where the plunger member comprises a plunger seal on a distal end of the plunger member and slidably located within the syringe body, the plunger member further comprising a proximal end comprising a groove for engagement within a plunger fitting of the dispensing unit.

13. The syringe of claim 10, where the proximal end of the plunger member comprises a tapered shape.

14. The syringe of claim 1, where the retaining member is integrally formed with the syringe body.

15. The syringe of claim 1, where the retaining member comprises a planar surface extending parallel to the axis of the syringe body.

16. The syringe of claim 1, where the retaining member comprises a thickness less than a diameter of the syringe body.

17. A dispensing system comprising:
a syringe comprising:
a syringe body comprising a reservoir and plunger slidably located therein;
a retaining member comprising a planar surface extending along a length of the syringe body and extending away from an axis of the syringe body, a retention surface formed by a slot extending entirely through the planar surface, the retaining member configured to distribute a force applied to the retention surface along the length of the syringe body to reduce distortion of the syringe body; and
a housing assembly comprising:
a moveable locking surface, the locking surface enlargeable against the retention surface to releasably lock the retaining member to the housing assembly to prevent movement of the syringe body relative to the housing assembly;
a drive mechanism coupled to the housing assembly, the drive mechanism comprising a plunger fitting configured to receive a portion of the plunger;
an actuator; and
a drive assembly coupled to the actuator, such that movement of the actuator in a single continuous stroke moves the plunger fitting relative to the distal end of the housing.

18. (canceled)

19. The dispensing unit of claim 17, where a portion of the retention surface is parallel to an axis of the syringe body.

20. The dispensing unit of claim 17, where the retaining member comprises a flange shape, the flange shape comprising at least one slot extending parallel to an axis of the syringe body, where at least a side of the at least one slot defines the retention surface.

21. The dispensing unit of claim 20, where the flange comprises a planar surface extending parallel to an axis of the syringe body.

22. The dispensing unit of claim 20, where the slot comprises an arcuate shape.

23. (canceled)

24. The syringe of claim 17 where the retaining member extends along at least a half of the length of the syringe body.

25. The syringe of claim 17, where the retaining member is detachable from the syringe body.

26. The syringe of claim 25, where the retaining member is integrally formed with the syringe body and detached through a weakened area.

27. The syringe of claim 17, further comprising a luer fitting at a distal end of the syringe body.

28. The syringe of claim 17, further comprising a plunger member slidably positioned within the syringe body.

29. The syringe of claim 28, where the plunger member comprises a plunger seal on a distal end of the plunger member and slidably located within the syringe body, the plunger member further comprising a proximal end comprising a groove for engagement within a plunger fitting of the dispensing unit.

30. The syringe of claim 29, where the proximal end of the plunger member comprises a tapered shape.

31.-34. (canceled)

32. A dispensing system comprising:
a syringe comprising:
a syringe body comprising a reservoir and plunger slidably located therein;
a retaining member extending along a length of the syringe body and extending away from an axis of the syringe body, the retaining member comprising an opening having a retention surface, the retaining member configured to distribute a force applied to the retention surface along the length of the syringe body to reduce distortion of the syringe body, where the retaining member comprises a flange shape comprising a plurality of slots, where at least one of the plurality of slots extends parallel to an axis of the syringe body and has a side that defines the retention surface; and
a housing assembly comprising:
a moveable locking surface, the locking surface enlargeable against the retention surface to releasably lock the retaining member to the housing assembly to prevent movement of the syringe body relative to the housing assembly;
a drive mechanism coupled to the housing assembly, the drive mechanism comprising a plunger fitting configured to receive a portion of the plunger;
an actuator; and
a drive assembly coupled to the actuator, such that movement of the actuator in a single continuous stroke moves the plunger fitting relative to the distal end of the housing.
